(19) United States
(12) Patent Application Publication
(10) Pub. No.: US 2005/0191359 A1
(43) Pub. Date: Sep. 1, 2005

(54) Water soluble nanoparticles and method for their production

(75) Inventors: Rina Goldshtein, Har Hebron (IL); Roman Kamburg, Rishon Lezion (IL); Galina Ratner, Rehovot (IL); Michael Kopylov, Beer Sheva (IL); Ilya Zelkind, Ofakim (IL); Vadim Goldshtein, Har Hebron (IL); Olga Skylarsky, Rehovot (IL); Boris Tulbovich, Ashkelon (IL); Erwin Stern, Raanana (IL)

Correspondence Address: BROWDY AND NEIMARK, P.L.L.C.;624 NINTH STREET, NW, SUITE 300, WASHINGTON, DC, 20001-5303, US

(73) Assignee: SoluBest Ltd., Rehovot (IL)

(21) Appl. No.: 10/952,380

(22) Filed: Sep. 29, 2004

Related U.S. Application Data
(63) Continuation-in-part of application No. 10/256,023, filed on Sep. 26, 2002.
Application No. 10/256,023 is a continuation-in-part of application No. 09/966,847, filed on Sep. 28, 2001, now Pat. No. 6,878,693.
(60) Provisional application No. 60/507,623, filed on Sep. 30, 2003.

(51) Int. Cl. A61K 31/7048; A61K 31/445; A61K 31/337; A61K 9/14

(52) U.S. Cl. 424/489; 514/28; 514/319; 514/449

(57) ABSTRACT

Hydrophilic dispersions of stable nano-sized particles are provided comprising: (a) a water-insoluble or water-soluble active compound, wherein said active compound is selected from the group consisting of a macrolide antibiotic, donepezil hydrochloride, an azole compound and a taxane; and (b) an amphiphilic polymer which wraps said active compound in a non-crystalline manner to form a nano-sized molecular entity in which no valent bonds are formed.

### CROSS-REFERENCE TO RELATED APPLICATIONS

The present application is a continuation-in-part of application Ser. No. 10/256,023, filed Sep. 26, 2002, which is a continuation-in-part of application Ser. No. 09/966,847, filed Sep. 28, 2001, and is a non-provisional of the Provisional Application No. 60/507,623, filed Sep. 30, 2003, the entire contents of each and all these applications being hereby incorporated by reference herein in their entirety as if fully disclosed herein.

### FIELD OF THE INVENTION

The present invention is in the field of nanoparticles. More particularly, the invention relates to soluble nano-sized particles (hereinafter “solu-nanoparticles”) consisting of inclusion complexes of active compounds such as pharmaceutical drugs or pesticides surrounded by and entrapped within suitable amphiphilic polymers, and methods of producing said solu-nanoparticles.

### BACKGROUND OF THE INVENTION

Two formidable barriers to effective drug delivery and hence to disease treatment, are solubility and stability. To be absorbed in the human body, a compound has to be soluble in both water and fats (lipids). Solubility in water is, however, often associated with poor fat solubility and vice-versa.

Over one third of drugs listed in the U.S. Pharmacopoeia and about 50% of new chemical entities (NCEs) are insoluble or poorly insoluble in water. Over 40% of drug molecules and drug compounds are insoluble in the human body. In spite of this, lipophilic drug substances having low water solubility are a growing drug class having increasing applicability in a variety of therapeutic areas and for a variety of pathologies. There are over 2500 large molecules in various stages of development today, and over 5500 small molecules in development (See Drug Delivery Companies Report 2001, p.2, www.pharmaventures.com). Each of the existing companies focusing on these large and small molecules has its own restriction and limitations with regard to both large and small molecules on which they focus.

Solubility and stability issues are major formulation obstacles hindering the development of therapeutic agents. Aqueous solubility is a necessary but frequently elusive property for formulations of the complex organic structures found in pharmaceuticals. Traditional formulation systems for very insoluble drugs have involved a combination of organic solvents, surfactants and extreme pH conditions. These formulations are often irritating to the patient and may cause adverse reactions. At times, these methods are inadequate for solubilizing enough of a quantity of a drug for a parenteral formulation. In such cases, doctors may administer an “overdosage”, such as for example with poorly soluble vitamins. In most cases, this overdosage does not cause any harm since the unabsorbed quantities exit the body with urine. Overdosage does, however, waste a large amount of the active compound.

The size of the drug molecules also plays a major role in their solubility and stability as well as bioavailability. Bioavailability refers to the degree to which a drug becomes available to the target tissue or any alternative in vivo target (i.e., receptors, tumors, etc.) after being administered to the body. Poor bioavailability is a significant problem encountered in the development of pharmaceutical compositions, particularly those containing an active ingredient that is poorly soluble in water. Poorly water-soluble drugs tend to be eliminated from the gastrointestinal tract before being absorbed into the circulation. It is known that the rate of dissolution of a particulate drug can increase with increasing surface area, that is, decreasing particle size

Recently, there has been an explosion of interest in nanotechnology, the manipulation on the nanoscale. Nanotechnology is not an entirely new field: colloidal sols and supported platinum catalysts are nanoparticles. Nevertheless, the recent interest in the nanoscale has produced, among numerous other things, materials used for and in drug delivery. Nanoparticles are generally considered to be solids whose diameter varies between 1-1000 nm.

Although a number of solubilization technologies do exist, such as liposomes, cylcodextrins, microencapuslation, and dendrimers, each of these technologies has a number of significant disadvantages.

Phospholipids exposed to aqueous environment form a bi-layer structure called liposomes. Liposomes are microscopic spherical structures composed of phospholipids that were first discovered in the early 1960s. In aqueous media, phospholipid molecules, being amphiphilic, spontaneously organize themselves in self-closed bilayers as a result of hydrophilic and hydrophobic interactions. The resulting vesicles, referred to as liposomes, therefore encapsulate in the interior part of the aqueous medium in which they are suspended, a property that makes them potential carriers for biologically active hydrophilic molecules and drugs in vivo. Lipophilic agents may also be transported, embedded in the liposomal membrane. Liposomes resemble the bio-membranes and have been used for many years as a tool for solubilization of biological active molecules insoluble in water. They are non-toxic and biodegradable and can be used for specific target organs.

Liposome technology allows for the preparation of smaller to larger vesicles, using unilamellar (ULV) and multilamellar (MLV) vesicles. MLVs are produced by mechanical agitation. Large ULVs are prepared from MLV by extrusion under pressure through membranes of known pore size. The sizes are usually 200 nm or less in diameter; however, liposomes can be custom designed for almost any need by varying lipid content, surface change and method of preparation.

As drug carriers, liposomes have several potential advantages, including the ability to carry a significant amount of drug, relative ease of preparation, and low toxicity if natural lipids are used. However, common problems encountered with liposomes include: low stability, short shelf-life, poor tissue specificity, and toxicity with non-native lipids. Additionally, the uptake by phagocytic cells reduces circulation times. Furthermore, preparing liposome formulations that exhibit narrow size distribution has been a formidable challenge under demanding conditions, as well as a costly one. Also, membrane clogging often results during the production of larger volumes required for pharmaceutical production of a particular drug.

Cyclodextrins are crystalline, water-soluble, cyclic, non-reducing oligo-saccharides built from six, seven, or eight glucopyranose units, referred to as alpha, beta and gamma cyclodextrin, respectively, which have long been known as products that are capable of forming inclusion complexes. The cyclodextrin structure provides a molecule shaped like a segment of a hollow cone with an exterior hydrophilic surface and interior hydrophobic cavity.

The hydrophilic surface generates good water solubility for the cyclodextrin and the hydrophobic cavity provides a favorable environment in which to enclose, envelope or entrap the drug molecule. This association isolates the drug from the aqueous solvent and may increase the drug's water solubility and stability. For a long time most cyclodextrins had been no more than scientific curiosities due to their limited availability and high price.

As a result of intensive research and advances in enzyme technology, cyclodextrins and their chemically modified derivatives are now available commercially, generating a new technology: packing on the molecular level. Cyclodextrins are, however, fraught with disadvantages. An ideal cyclodextrin would exhibit both oral and systemic safety. It would have water solubility greater than the parent cyclodextrins yet retain or surpass their complexation characteristics. The disadvantages of the cyclodextrins, however, include: limited space available for the active molecule to be entrapped inside the core, lack of pure stability of the complex, limited availability in the marketplace, and high price.

Microencapsulation is a process by which tiny parcels of a gas, liquid, or solid active ingredient (also referred to herein and used interchangeably with “core material”) are packaged within a second material for the purpose of shielding the active ingredient from the surrounding environment. These capsules, which range in size from one micron (one-thousandth of a millimeter) to approximately seven millimeters, release their contents at a later time by means appropriate to the application.

There are four typical mechanisms by which the core material is released from a microcapsule: (1) mechanical rupture of the capsule wall, (2) dissolution of the wall, (3) melting of the wall, and (4) diffusion through the wall. Less common release mechanisms include ablation (slow erosion of the shell) and biodegradation.

Microencapsulation covers several technologies, where a certain material is coated to obtain a micro-package of the active compound. The coating is performed to stabilize the material, for taste masking, preparing free flowing material of otherwise clogging agents etc. and many other purposes. This technology has been successfully applied in the feed additive industry and to agriculture. The relatively high production cost needed for many of the formulations is, however, a significant disadvantage.

In the cases of nanoencapsulation and nanoparticles (which are advantageously shaped as spheres and, hence, nanospheres), two types of systems having different inner structures are possible: (i) a matrix-type system composed of an entanglement of oligomer or polymer units, defined as nanoparticles or nanospheres, and (ii) a reservoir-type system, consisting of an oily core surrounded by a polymer wall, defined as a nanocapsule.

Depending upon the nature of the materials used to prepare the nanospheres, the following classification exists: (a) amphiphilic macromolecules that undergo a cross-linking reaction during preparation of the nanospheres; (b) monomers that polymerize during preparation of the nanoparticles; and (c) hydrophobic polymers, which are initially dissolved in organic solvents and then precipitated under controlled conditions to produce nanoparticles.

Problems associated with the use of polymers in micro- and nanoencapsulation include: the use of toxic emulgators in emulsions or dispersions, polymerization or the application of high shear forces during emulsification process, insufficient biocompatibility and biodegrability, balance of hydrophilic and hydrophobic moieties, etc. These characteristics lead to insufficient drug release.

Dendrimers are a class of polymers distinguished by their highly branched, tree-like structures. They are synthesized in an iterative fashion from ABn monomers, with each iteration adding a layer or “generation” to the growing polymer. Dendrimers of up to ten generations have been synthesized with molecular weights in excess of 106 kDa. One important feature of dendrimeric polymers is their narrow molecular weight distributions. Indeed, depending on the synthetic strategy used, dendrimers with molecular weights in excess of 20 kDa can be made as single compounds.

Dendrimers, like liposomes, display the property of encapsulation, and are able to sequester molecules within the interior spaces. Because they are single molecules, not assemblies, drug-dendrimer complexes are expected to be significantly more stable than liposomal drugs. Dendrimers are thus considered as one of the most promising vehicles for drug delivery systems. However, the dendrimer technology is still in the research stage, and it is speculated that it will take years before it is applied in the industry as a safe and efficient drug delivery system.

What is needed is a safe, biocompatible, stable and efficient drug delivery system that comprises nano-sized particles of an active ingredient for enhanced bioavailability and which overcomes the problems inherent in the prior art.

### SUMMARY OF THE INVENTION

Lipophilic and hydrophilic compounds that are solubilized in the form of nano-sized particles, or “nanoparticles”, can be used in pharmacology, in the production of food additives, cosmetics, and agriculture, as well as in pet foods and veterinary products, amongst other uses.

The present invention provides nanoparticles and methods for the production of soluble nanoparticles and, in particular, inclusion complexes of water-insoluble lipophilic and water-soluble hydrophilic organic materials.

Soluble nanoparticles, referred to as “solu-nanoparticles” in accordance with the present invention, are differentiated by the use of water soluble amphiphilic polymers that are capable of producing molecular complexes with lipophilic and hydrophilic active compounds or molecules (particularly, drugs and pharmaceuticals). The solu-nanoparticles formed in accordance with the present invention render insoluble compounds soluble in water and readily bioavailable in the human body.

The active compound may be a water-insoluble lipophilic or a water-soluble hydrophilic organic compound. The water-soluble amphiphilic polymers used are capable of producing molecular complexes with the lipophilic or hydrophilic active compounds and the solu-nanoparticles formed in accordance with the present invention render insoluble compounds soluble in water and readily bioavailable in the human body.

An inclusion complex, by definition, is a complex in which one component, designated “the host”, forms a cavity in which molecular entities of a second chemical species, designated “the guest”, are located. Thus, in accordance with the present invention, it can be defined that the solu-nanoparticles comprise inclusion complexes in which the host is the amphiphilic polymer or group of polymers and the guest is the active compound molecules wrapped and fixated or secured within the cavity or space formed by said polymer host.

In accordance with the present invention, the inclusion complexes contain the active compound molecules, which interact with the polymer by non-valent interactions and form a polymer-active compound as a distinct molecular entity. A significant advantage and unique feature of the inclusion complex of the present invention is that no new chemical bonds are formed and no existing bonds are destroyed during the formation of the inclusion complex. The particles comprising the inclusion complexes are nano-level in size, and no change occurs in the drug molecule itself when it is enveloped, or advantageously wrapped, by the polymer.

The outer surface of the inclusion complexes is comprised of a polymer that carries the active compound, when it is a drug molecule, to the target destination. Depending upon the polymer used in the formation of the solu-nanoparticles, the drugs and pharmaceuticals within the complex are able to reach specific areas of the body readily and quickly. The polymer and active compound selected will also provide solu-nanoparticles capable of multi-level, multi-stage and/or controlled release of the drug or pharmaceutical within the body.

The solu-nanoparticles of the invention remain stable for long periods of time, may be manufactured at a low cost, and may improve the overall bioavailability of the active compound.

In particular, the present invention provides water-soluble and stable nano-sized particles comprising hydrophilic inclusion complexes consisting essentially of an active compound surrounded by and entrapped within an amphiphilic polymer, wherein said active compound is in a non-crystalline state and said inclusion complex is stabilized by non-valent interactions between the active compound and the surrounding amphiphilic polymer, and wherein said inclusion complex is selected from the group consisting of:
(i) an inclusion complex wherein the active compound is a macrolide antibiotic and the amphiphilic polymer is a polysaccharide;
(i) an inclusion complex wherein the active compound is donepezil hydrochloride and the amphiphilic polymer is a polysaccharide;
(iii) an inclusion complex wherein the active compound is an azole fungicide and the amphiphilic polymer is selected from the group consisting of a polysaccharide, polyacrylic acid, a copolymer of polyacrylic acid, polymethacrylic acid and a copolymer of polymethacrylic acid; and
(iv) an inclusion complex wherein the active compound is a taxane and the amphiphilic polymer is gelatin.

The above description sets forth rather broadly the more important features of the present invention in order that the detailed description thereof that follows may be better understood, and in order that the present contributions to the art may be better appreciated. Other objects and features of the present invention will become apparent from the following detailed description considered in conjunction with the accompanying drawings. It is to be understood, however, that the drawings are designed solely for the purposes of illustration and not as a definition of the limits of the invention, for which reference should be made to the appended claims.

### BRIEF DESCRIPTION OF THE DRAWINGS

The invention will be better understood by reference to the appended figures in which:

FIG. 1 illustrates in vitro antibacterial (Micrococcus luteus) activity of two concentrations of commercial (clarithro) and nano-particles of clarithromycin-starch inclusion complexes observed until 216 hours post-application.

FIG. 2 illustrates apparent plasma levels in rats orally administered with clarithromycin-starch in nano-particle complex (SOLUCLARI) or with commercial clarithromycin.

FIG. 3 is an SEM micrograph illustrating the consistent spherical particles of a clarithromycin-starch inclusion complex (#IC-75, Table 3).

FIG. 4 illustrates the comparison of the solubility of erythromycin and clarithromycin alone and as part of inclusion complexes with starch.

FIG. 5 illustrates the X-ray diffraction comparison of intact erythromycin with the inclusion complex of erythromycin-starch.

FIG. 6 illustrates the X-ray diffraction comparison of intact clarithromycin with the inclusion complex of clarithromycin-starch.

FIGS. 7A-7B illustrate X-ray spectra of 6-month old clarithromycin-starch inclusion complex sample (bottom trace) compared to the commercially available clarithromycin (upper trace) (7A) and of 10-month old azithromycin-chitosan inclusion complex sample (bottom trace) compared to the commercially available azithromycin (upper trace) (7B).

FIG. 8 illustrates the size distribution of nano-particles comprising clarithromycin-chitosan inclusion complexes (#10-134, Table 3) having a size of approximately 838 nm, as measured by light diffraction (ALV).

FIG. 9 illustrates the in vitro release via a dialysis membrane of commercial clarithromycin (Clari) in comparison with particles comprising clarithromycin inclusion complexes with PVA (S-Clari#-34, Table 3) or nano-particles comprising clarithromycin inclusion complexes with chitosan (S-Clari#135, Table 3).

FIG. 10 illustrates the size distribution of nano-particles comprising azithromycin-chitosan inclusion complexes (#10-148/2, Table 4) having a size of approximately 362 nm, as measured by light diffraction (ALV).

FIG. 11 illustrates the size distribution of nano-particles comprising itraconazole-modified starch inclusion complexes (#23-120, Table 5) having a size of approximately 414 nm, as measured by light diffraction (ALV).

FIGS. 12A-12B illustrates differential scanning calorimetry (DSC) analysis of commercial crystalline itraconazole (12A) and of nano-particles comprising itraconazole-polyacrylic acid inclusion complexes (#IT-56, Table 5).

FIG. 13 illustrates the size distribution of nano-particles comprising taxol-gelatin inclusion complexes (# 25-85, Table 6) having a size of approximately 179 nm, as measured by light diffraction (ALV).

FIG. 14 illustrates the size distribution of nano-particles comprising donepezil-modified starch inclusion complexes (#LG-7-51, Table 7) having a size of approximately 600 nm, as measured by light diffraction (ALV).

FIG. 15 illustrates oral absorption of the following materials in a preclinical model involving rats: commercial formulation of azithromycin (Azenil), fluid formulations of nano-particles of azithromycin-chitosan inclusion complexes (from lots 28-39 and 28-59) and of lot 28-59 that were further formulated in tablet form.

### DETAILED DESCRIPTION OF THE INVENTION

The nanoparticles of the present invention comprise the insoluble or soluble active compound or core, wrapped within a water-soluble amphiphilic polymer. A variety of different polymers can be used according to the present invention for any of the selected active compound, that can be lipophilic or hydrophilic. The polymer, or groups of polymers, is selected according to an algorithm that takes into account various physical properties of both the active lipophilic or hydrophilic compound and the interaction of this compound within the resulting active compound /polymer nano-soluparticle.

More particularly, the ingredients of the composition of the present invention comprise the active (hydrophilic or, preferably, lipophilic) compound and the polymer to provide a molecular entity. The active compound may be any organic molecule or compound and may be preferably a drug or pharmaceutical composition. The active compound can be small or large, simple or complex, heavy or light and may comprise a variety of functional groups. The polymer or polymers used to make up the complex may be selected from the group of polymers approved for human use (i.e. biocompatible). Such polymers comprise, for example, but are not limited to: polysaccharides, e.g. starch, alginate and chitosan, polyacrylic acid and its derivatives and copolymers thereof, polyethylene imine and its derivatives, polymethacrylic acid and its derivatives and copolymers thereof, polyethylene oxide and its derivatives, polyvinyl alcohol and its derivatives, polyisoprene derivatives, polybutadiene derivatives, and gelatin.

As recited, the polymer or groups of polymers used in the formation of the nano-soluparticles of the present invention are selected according to an algorithm that takes into account various physical properties of the active compounds and the polymer or polymers, as well as their future interaction in the resulting complex. The algorithm is utilized in this manner to select the optimal polymer(s) and to assess processing parameters such as pH, ionic force, temperature and various solvent parameters. More specifically, the amphiphilic polymer is selected using the algorithm that assesses the molecular weight, dimensions (in three directions) and the solubility of the compound in aqueous solvents. The algorithm also takes into consideration the following properties of the polymer itself in selecting a polymer for the active molecule/polymer interaction in the formation of the complex: molecular weight, basic polymer chain length, the length of the kinetic unit, the solubility of the polymer in water, the overall degree of solubility, the degree of polymer flexibility, the hydrophilic-lipophilic balance (HLB), and the polarity of the hydrophilic groups of the polymer. The main properties of the polymer include its HLB, the length and the flexibility of its polymer chain, and also the state of polarity of the hydrophilic groups.

One important parameter in the choice of the polymer or polymers is the HLB, i.e., the measure of the molecular balance of the hydrophilic and lipophilic portions of the compound. Within the HLB International Scale of 0-20, lipophilic molecules have a HLB of less than 6, and hydrophilic molecules have a HLB of more than 6. Thus, according to the present invention, the HLB of the polymer is selected in such a way that, after combining to it the active compound, the total resulting HLB value of the complex will be greater than 8, rendering the complex water-soluble.

Following the selection of the active compound, a determination is made of its requisite properties for construction of a geometrical model and a polymer suitable for complexation with the given compound is then selected. At this stage, a geometrical model of the complex is constructed and determination is made of the length of the fragment of the polymer chain needed for the complex. The HLB is calculated following the building of a virtual complex on a computer screen. To this end, existing computer programs for simulation of molecular structures are used. The HLB can be calculated as a ratio of hydrophilic and lipophilic groups of the virtual complex. The molecular weight of the complex is easily computed and its geometry is determined. More precisely, total HLB of the complex in accordance with the present invention can be calculated after the virtual construction of the complex on the computer screen of a computer system upon which the suitable algorithm has been loaded as software. The algorithm that determines the summary HLB thus plays a major role in the selection of components from which the complex is formed. The parameters and library information pertaining to active compounds and polymer molecules are stored in the computer program for calculation of the summary HLB of the complex to be formed.

For the generation of the geometric model, a determination of the weight correlation of the “amphiphilic polymer to active molecule” may be made based on the total length of the polymer chain, length of the fragment needed to create the complex, molecular mass of the active compound and molecular mass of the fragment, according to the following formula:

$${N}_{c} = \frac{{M}_{f} \times {N}_{f}}{{M}_{1}} = \frac{{m}_{F}}{{M}_{1}} \times \frac{{M}_{p}}{{M}_{f}} = \frac{{M}_{p} ( g \text{-} mol )}{{M}_{1} ( g \text{-} mol )}$$

wherein N_{c} is the weight ratio of the “amphiphilic polymer to lipophilic compound”; M_{f} is the molecular mass of the polymer fragment; Mₗ is the molecular mass of the lipophilic compound; Mₚ is the molecular mass of the polymer; and N_{f} is the quantity of the polymer fragments capable of participating in the complex creation.

Next, the physical parameters of the water solvent for the polymer are evaluated. At this stage, determination is made of the pH required to create the complex, the necessary ionic force and the required carrier for the active compound. Use of the above components creates optimal conditions for controlling the flexibility of the polymer chain.

The non-aqueous carrier solvent is then selected. The purpose of this solvent is to transfer the active compound into a very weak (low concentration) solution such that the molecules of the dissolved compound practically do not react with one another. This solution is then delivered into the reaction zone in the chemical reactor (discussed in detail in the parent U.S. application Ser. No. 09/966,847) for the creation of nano-dispersions, such as a nano-emulsion (having a liquid core material) or nano-suspension (having a solid core material). As used herein, the term “suspension” generally refers to a dispersion of fine particles in a liquid and the term “emulsion” generally refers to a mixture of two normally unmixable liquids in which one is colloidally suspended in the other (defining a dispersed phase). The particle sizes of the dispersed phase in an emulsion generally lie between a few hundred nanometers and a few tens of micrometers.

Unlike known processes for the preparation of nano-sized particles where polymers are used for stabilization of the dispersion formed, only some of the aforementioned amphiphilic polymers (with previously calculated hydrophilic-lipophilic balance HLB) are used in these dispersion stabilizations. Additionally, specific conditions are selected for the dynamic three-dimensional conformation of the amphiphilic polymer in the dispersion, which serves as the creator of the complex and fixator of the core active compound, as opposed to acting as a viscosifier (i.e., for increasing the viscosity). Previously calculated HLB provides for the necessary solubilization of the active compound.

Specific conditions created for the amphiphilic polymer in the “nano-dispersion” formation, result in two factors: (1) the provision of free rotation of the kinetic segments of the polymer chain around the chemical bonds, thus connecting these segments, and (2) the provision of non-valent interaction of the lipophilic functional groups of the amphiphilic polymer and the lipophilic groups of the compound intended for solubilization. These specific conditions include: the pH parameter of the dispersive medium, the ionic forces of the dispersive medium, the components composition of the dispersive medium, the temperature of the complex formulation, the process duration, and the mechanical components of the process. Each of these specific conditions will be discussed in more detail below.

The pH parameter of the dispersion medium: If the composition of the amphiphilic polymer includes ionogenic functional groups, the polymer could be soluble or at a pH higher than the isoelectric point (polyacids) or lower than isoelectric point (polybases) depending on the polarity of these groups. In both of these cases, the isoelectric point could be determined with a high degree of accuracy on the curve of “viscosity of the polymer solution-pH of the polymer solution”. These two types of polymers could participate in the complex creation only within the pH range where their solutions are viscous liquids. For polymers with non-ionogenic functional groups, the clearly defined isoelectric point does not exist and for this reason these polymers could participate in the complex creation in a wide pH range.

Ionic force of the dispersive medium: Under the influence of the ions of the water-soluble salts in the polymer solution, the geometry of the amphiphilic polymer chains changes. This factor is used for creation of stereospecific conditions of non-covalent interaction between lipophilic groups of the polymer and the lipophilic itself. Nonetheless, many polymers react so actively on the appearance of the salts (a “salting out” process of the polymer), that it is not always possible to utilize this factor in the reaction of complex creation.

Competition exists between the ions and the polymer for water molecules and the ions take water from the hydrate shells of the polymer. As a result of decreasing hydrate shell, the polymer coils to a globule. The greater the ionic activity, the greater is the polymer coiling to the globule.

Components composition of the dispersive medium: With the help of the composition of the solvents, it is possible to flexibly control the geometry of the macromolecules. However, for the purpose of solubility (solubilization) of pharmaceuticals, food additives and cosmetics compounds, only biologically safe solvents such as, but not limited to, ethyl acetate, methyl acetate, glycerol, ethylene glycol and less often ethyl alcohol, isobutanol and dimethylsulfoxide should be used.

Temperature of the complex formation: With the changes of the temperature of the polymer solution, the hydration conditions of the polymer molecule, and accordingly its configuration in the solution, drastically changes. With the raising of the temperature, hydration shells surrounding the polymer molecule start to detach and the linear macromolecule starts to take on globular form. At the same time, the flexibility of the macromolecule increases. As a result, additional positive conditions for complex creation are created.

The process duration: Because of the non-valent interaction during creation of the inclusion complex, the limiting phase of the process consists of the diffusion of the active, e.g. lipophilic, compounds and the macromolecules to each other; for each reaction system exists a minimum time for complex creation. If less time is allowed, the system remains two-phased. This two-phased nano-dispersion is thermodynamically unstable. In the next step, the carrier/organic solvent is evaporated, the polymer molecules in the solution then cover and entrap molecules of the active compounds, creating stable nano-particles of the dispersed phase in sizes ranging from 1-1000 nm.

The mechanical component of the process: Mixers, dispersers, homogenizers and other equipment are employed to provide maximum dispersing of the active compound in the water-polymer solution and accelerate creation formation of an emulsion or suspension with nano-dimension sized particles in a dispersed phase. This equipment may form part of a chemical reactor as described in the parent U.S. application Ser. No. 09/966,847, or any other suitable chemical reactor may be constructed to form the soluble nano-particles of the invention.

The combined effect of the above conditions aids in achieving specifically selected dimensions and proportions for the complex, the maximum dispersing of the active compound and the optimal conditions for the non-valent interaction of the polymer and these compounds during complex formation.

As recited above, the preparation of the inclusion complexes in accordance with the present invention requires a number of calculations and procedures to be performed prior to commencing the process of preparing the complex. Some calculations and procedures, which are determined using an algorithm on a computer system, include:
(a) calculating the composition and properties of the components for preparing the complex, which comprises an active compound, an amphiphilic polymer, and carrier solvent;
(b) calculating the weight ratio of the amphiphilic polymer to the active compound;
(c) evaluating the physical parameters of the water solvent for the amphiphilic polymer;
(d) determining the proper non-aqueous solvent;
(e) creating a geometric model of the complex.

The algorithm is not limited to these calculations and may be programmed to make additional calculations and determinations as necessary depending upon the properties and characteristics of the complex to be made.

As recited, the production of the molecular complex consisting of an active compound and an amphiphilic polymer according to the present invention, requires the dispersal of the active compound to nano-particle size. The nano-sized particles assure an almost immediate interaction between the dispersed nano-sized particles of the active compound and the polymer molecules. In accordance with the process of the invention, it is also necessary to prevent reverse aggregation (coacervation) of the nano-particles, and to assure an immediate interaction between the dispersed nano-particles of the active compound and the polymer molecules. This assures the formation of a stable complex (inclusion or other). The size of the active compound is determined by constructing its geometrical model (taking into account length of the connections and angles between these connections), and thereafter transferring the compound into a spherical configuration or other geometric shapes. The diameter of this sphere is the deciding measuring size of the active compound. There is a need to take into account that lipophilic compounds with long chain structures, as a rule, assume a shape having a globular configuration.

In accordance with the present invention, during the process of forming the soluble nano-sized particles or “solu-nanoparticles”, a polymer is added to an aqueous solvent, preferably water, to form a polymer solution in a first vessel of a chemical reactor. Additionally, ingredients may be added to adjust the pH and ionic force level of this solution as needed based on the parameters determined via the algorithm used to select the active compound and polymer. The active compound, which may be a water-insoluble (lipophilic) or a water-soluble (hydrophilic) compound, is placed in a second vessel of the chemical reactor. The active compound (or core) may be of any size, dimension or weight, and may comprise any of a variety of functional groups. A solution of the active lipophilic or hydrophilic compound in a non-aqueous solvent (or mixture of solvents) is referred to as the “carrier”. The velocity of pouring or adding the carrier to the polymer solution is regulated by one or more regulating taps, which ensure that the lipophil solution being added to the polymer solution has a concentration below 3%.

The active compound solution is formed when the polymer solution is heated and steam from the heated polymer solution condenses and dissolves the active compound, present in the second vessel. The active compound solution (in carrier) is then mixed with the polymer solution to form a dispersed phase in emulsion or suspension. Within the chemical reactor, the emulsion is fed into an area of turbulence caused by a disperser (more precisely a nano-disperser) that causes the formation of nano-sized active compound molecules within the emulsion or suspension. The area of turbulence is referred to as the “action zone” or the “zone of interaction”. The emulsion or suspension being fed into the area of turbulence has a Reynolds number of Re>10,000. The emulsion thus becomes a “nano-emulsion” or “nano-suspension” having particles in the range of approximately 1 to approximately 1000 nm. The particle production can also be extended to include small micron-sized particles and these particles may be suitable for several uses and are also encompassed by the present invention. Within the nano-emulsion or nano-suspension there exists a dispersion medium comprised of the polymer solution, and a dispersed phase comprising the solution of the active compound in the carrier. This two-phased nano-emulsion or nano-suspension is, however, unstable. Evaporating the carrier leaves particles of the dispersed phase in sizes ranging from approximately 1 to approximately 1000 nanometers. The polymer molecule in the polymer solution then surrounds or envelopes, and more appropriately wraps, the active compounds that had remained in the particles of the dispersed phase after evaporation of the carrier, thus forming a homogeneous nano-sized dispersion of water-insoluble lipophilic compound wrapped by a hydrophilic polymer in an inclusion complex. The remaining carrier is then evacuated by vacuum evaporation or other appropriate drying techniques (e.g., lyophilization, vacuum distillation). As a result of the algorithm used to select the optimal active compound and polymer for the formation of the emulsion or suspension and resulting complex, no free polymer generally remains after the evaporation of the carrier. Following evaporation of the carrier, the stable inclusion complex is comprised of amorphous and/or partially crystalline or crystalline active entities.

As used herein, the term “non-crystalline” refers to materials both in amorphous or disordered crystalline state. In preferred embodiments, the material is amorphous. It is known by those skilled in the art that the amorphous state is preferred for drug delivery as it may indeed enhance bioavailability.

As used herein, the terms “water-soluble nano-particles”, “aqueous solution of nano-particles” and “nano-dispersion” are used interchangeably and both intend to refer to the same thing, namely, to a fine dispersion of the nano-particles that may have the appearance of a solution, but is not a classical aqueous solution.

As used herein, the terms “stable nano-dispersion” and “nano-dispersion of water-soluble and stable nano-sized particles” are used interchangeably and both intend to refer to the same thing, namely, to a stable fine dispersion of the nano-particles.

Stability of the nano-particles and of the inclusion complexes has more than one meaning. The nano-particles should be stable as part of a nanocomplex over time, while remaining in the dispersion media. The nano-dispersions are stable over time without separation of phases. Furthermore, the amorphous state should be also retained over time.

It is worth noting that in the process used in the present invention, the components of the system do not result in micelles nor do they form classical dispersion systems. The technology of the present invention causes the following:
(i) after forming the inclusion complex, the poorly soluble or insoluble (or even non-wettable) active compound becomes pseudo-soluble. When the particle size is about 20-30 nm, then the material becomes soluble and visually transparent, rather than opaque;
(ii) after dispersion of the active compound to nano-size and fixation by the polymers to form an inclusion complex, enhanced solubility in physiological fluids, in vivo, improved absorption, and improved biological activity, as well as transmission to a stable non-crystalline, preferably amorphous, state, are achieved;
(iii) a crystalline biologically-active compound becomes amorphous and thus exhibits improved biological activity.

In most preferred embodiments of the present invention, not less than 80% of the nano-particles in the nano-dispersion are within the size range, when the size deviation is not greater than 20%, and the particle size is within the nano range, namely less than 1000 nm.

In an advantageous and preferred embodiment of the invention, the polymer molecule in the polymer solution “wraps” the active compound via non-valent interactions. As used herein, the term “non-valent” is intended to refer to non-covalent, non-ionic and non-semi-polaric bonds and/or interactions, and includes, for example, electrostatic forces, Van der Waals forces, and hydrogen-bonds between the polymer and the active compound in the inclusion complex such that the non-valent interactions fixate the active compound within the polymer which thus reduces the molecular flexibility of the active compound and polymer. The formation of any valent bonds could change the characteristics or properties of the active compound. The formation of non-valent bonds preserves the structure and properties of the lipophilic compound, which is particularly important when the active compound is a pharmaceutical.

The process of the invention is useful for preparing aqueous solutions of nano-particles comprising inclusion complexes wherein the active compound is a pharmaceutical drug for human or veterinary use or an active compound for use in the agriculture or any other suitable technological area in which nano-particles of the type described herein may be needed or desired. In preferred embodiments, the active compound is a pharmaceutical drug selected from the classes of drugs such as, but not limited to, analgesics, anti-inflammatory agents, anthelmintics, antianginal agents, anti-arrhythmic agents, antibiotics (including penicillins, cephalosporins, macrolides), anticoagulants, antidepressants, antidiabetic agents, antiepileptics, antigonadotropins, antihistamines, antihypertensive agents, antimuscarinic agents, antimycobacterial agents, antineoplastic agents, immunosuppressants, antithyroid agents, antiviral agents, anti-neoplastic agents and chemotherapeutic agents, anxiolytic sedatives (hypnotics and neuroleptics), astringents, beta-adrenoceptor blocking agents, blood products and substitutes, cardiacinotropic agents, contrast media, corticosterioids, cough suppressants (expectorants and mucolytics), diagnostic agents, diagnostic imaging agents, diuretics, dopaminergics (antiparkinsonian agents), fungicidal agents, haemostatics, immunosuppressive cyclic oligopeptides, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anorexics, sympathomimetics, thyroid agents, vasidilators and xanthines. Preferred drug substances include those intended for oral administration, intravenous administration, mucosal administration and pulmonary administration.

As recited, in a more preferred embodiment, the present invention provides a nano-dispersion of water-soluble and stable nano-sized particles comprising hydrophilic inclusion complexes consisting essentially of an active compound surrounded by and entrapped within an amphiphilic polymer, wherein said active compound is in a non-crystalline state and said inclusion complex is stabilized by non-valent interactions between the active compound and the surrounding amphiphilic polymer, and wherein said inclusion complex is selected from the group consisting of:
(i) an inclusion complex wherein the active compound is a macrolide antibiotic and the amphiphilic polymer is a polysaccharide or polyvinyl alcohol;
(ii) an inclusion complex wherein the active compound is donepezil hydrochloride and the amphiphilic polymer is a polysaccharide;
(iii) an inclusion complex wherein the active compound is an azole compound and the amphiphilic polymer is selected from the group consisting of a polysaccharide, polyacrylic acid, a copolymer of polyacrylic acid, polymethacrylic acid and a copolymer of polymethacrylic acid; and
(iv) an inclusion complex wherein the active compound is a taxane and the amphiphilic polymer is gelatin.

In one preferred embodiment, the nano-particles of the invention comprise inclusion complexes in which the active compound is a macrolide antibiotic such as, but not limited to, erythromycin and semi-synthetic derivatives thereof including clarithromycin, and the first azalide antibiotic, azithromycin. All these macrolide are large, lipophilic molecules, broad-spectrum antibiotics active against a wide variety of bacteria and can be used both in human and veterinary medicine. Macrolide antibiotics are particularly useful in treating respiratory infections.

Polymers suitable for the preparation of inclusion complexes with the macrolide antibiotics are polysaccharides, in natural form or modified. In one embodiment, the polysaccharide is starch that should preferably have a large proportion of linear chains, i.e. starch with high contents of amylose, the constituent of starch in which anhydroglucose units are linked by (-D-1,4 glucosidic bonds to form linear chains, and low contents of amylopectin, a constituent of starch having a polymeric, branched structure. The levels of amylose and amylopectin and their molecular weight vary between different starch types.

To improve its characteristics for use in the invention, starch, e.g. corn or potato starch, can be modified, for example by increasing its hydrophilicity by acid hydrolysis, e.g., with citric acid, and/or by reaction with an agent, e.g. polyethylene glycol (PEG) and/or hydrogen peroxide. In addition, starch can be subjected to thermal treatment, for example at 160-180° C., for about 30-60 min, to reduce the amount of branching, optionally after treatment with PEG and/or hydrogen peroxide (hereinafter designated “thermodestructed starch”)

Thus, in one preferred embodiment, the nano-particles of the invention comprise inclusion complexes in which the active compound is erythromycin and the amphiphilic polysaccharide is starch polymer selected from the group consisting of hydrolyzed starch, starch modified by different amounts of PEG, preferably PEG-400, and/or by H₂O₂, and thermodestructed starch.

In another preferred embodiment, the nano-particles of the invention comprise inclusion complexes in which the active compound is clarithromycin and the amphiphilic polysaccharide is selected from the group consisting of starch, chitosan and alginate, e.g. sodium alginate. The starch may be hydrolyzed starch, starch modified by different amounts of PEG, preferably PEG-400, and/or by H₂O₂, and thermodestructed starch.

In another preferred embodiment, the nano-particles of the invention comprise inclusion complexes in which the active compound is azithromycin and the amphiphilic polysaccharide is chitosan or an alginate derivative such as propylene glycol alginate (Manucol ester B). In another preferred embodiment, the, nano-particles of the invention comprise inclusion complexes in which the active compound is azithromycin and the amphiphilic polymer is polyvinyl alcohol (PVA).

In another preferred embodiment, the nano-particles of the invention comprise inclusion complexes in which the active compound is donepezil hydrochloride and the amphiphilic polymer is a polysaccharide.

Donepezil, 1-benzyl-4-((5,6-dimethoxy-1-indanon)-2-yl)methylpiperidine, and analogues, were described in U.S. Pat. No. 4,895,841 as acetylcholinesterase inhibitors and useful for treatment of various kinds of dementia including Alzheimer senile dementia, Huntington's chorea, Pick's disease, and ataxia. Donepezil hydrochloride is a white crystalline powder and is freely soluble in chloroform, soluble in water and in glacial acetic acid, slightly soluble in ethanol and in acetonitrile and practically insoluble in ethyl acetate and in n-hexane. Donepezil hydrochloride is available for oral administration in film-coated tablets containing 5 or 10 mg of donepezil hydrochloride for treatment of mild to moderate dementia of the Alzheimer's type. Amorphous donepezil hydrochloride is mentioned in the patents U.S. Pat. No. 5,985,864 and U.S. Pat. No. 6,140,321. Recently, U.S. Pat. No. 6,734,195 disclosed that wet granulation of donepezil hydrochloride yields, after drying and milling, a stable granulate that uniformly contains donepezil hydrochloride amorphous.

In accordance with the present invention, water-soluble nano-particles are provided comprising inclusion complexes in which the donepezil hydrocloride in a non-crystalline state, e.g. amorphous state, is wrapped by an amphiphilic polysaccharide and is fixated/stabilized by non-valent interactions with the surrounding amphiphilic polysaccharide. In one preferred embodiment, the polysaccharide is alginate. In another preferred embodiment, the polysaccharide is sodium starch glycolate. In still another embodiment, the polysaccharide is pregelatinized modified starch.

In another preferred embodiment, the nano-particles of the invention comprise inclusion complexes in which the active compound is an azole compound and the amphiphilic polymer is selected from the group consisting of a polysaccharide, polyacrylic acid, a copolymer of polyacrylic acid, polymethacrylic acid and a copolymer of polymethacrylic acid.

Azole compounds play a key role as antifungals in agriculture and in human mycoses and as nonsteroidal antiestrogens in the treatment of estrogen-responsive breast tumors in postmenopausal women. This broad use of azoles is based on their inhibition of certain pathways of steroidogenesis by high-affinity binding to the enzymes sterol 14-demethylase and aromatase. Azole fungicides show a broad antifungal activity and are used either to prevent fungal infections or to cure an infection. Therefore, they are important tools in integrated agricultural production. According to their chemical structure, azole compounds are classified into triazoles and imidazoles; however, their antifungal activity is due to the same molecular mechanism. Azole fungicides are broadly used in agriculture and in human and veterinary antimycotic therapies.

In accordance with the present invention, an “azole compound” refers to imidazole and triazole compounds for human or veterinary application or for use in the agriculture.

In one preferred embodiment, the azole compound is selected from azole fungicides used in many different antimycotic formulations including, but not limited to the triazoles terconazole, itraconazole, and fluconazole, and the imidazoles clotrimazole, miconazole, econazole, ketoconazole, tioconazole, isoconazole, oxiconazole, and fenticonazole.

In another embodiment, the azole compound is selected from azoles that act as nonsteroidal antiestrogens and can be used in the treatment of estrogen-responsive breast tumors in postmenopausal women, including, but not limited to letrozole, anastrozole, vorozole, and fadrozole.

In another embodiment, the azole compound is an azole fungicide useful in the agriculture including, but not limited to, the triazoles bitertanol, cyproconazole, difenoconazole, epoxiconazole, fluquinconazole, flusilazole, flutriafol, hexaconazole, metconazole, myclobutanil, penconazole, propiconazole, tebuconazole, triadimefon, triadimenol, and triticonazole, and the imidazoles imazalil, prochloraz, and triflumizole. In still another embodiment, the azole compound is a nonfungicidal azole for use in the agriculture such as the triazoles azocyclotin used as an acaricide, paclobutrazole as a growth regulator, carfentrazone as a herbicide, and isazophos as an insecticide, and the imidazole metazachlor used as herbicide.

In one more preferred embodiment, the azole compound is itraconazole, an azole medicine used to treat fungal infections. It is effective against a broad spectrum of fungi including dermatophytes (tinea infections), yeasts such as candida and malassezia infections, and systemic fungal infections such as histoplasma, aspergillus, coccidiodomycosis, chromo-blastomycosis. Itraconazole is available as 100 mg capsules under the trademark Sporanox™ (Janssen-Cilag). It is a white to slightly yellowish powder. It is lipophilic, insoluble in water, very slightly soluble in alcohols, and freely soluble in dichloromethane. Sporanox contains 100 mg of itraconazole coated on sugar spheres.

In one embodiment, the amphiphilic polymer used to wrap the azole compound is a polysaccharide, more preferably chitosan or hydrolyzed or thermodestructed starch, both optionally modified by PEG, H₂O₂ or both. Alginate can also be used with certain concentrations of the azole compound (see Table 5 hereinbelow).

In another embodiment, the amphiphilic polymer used to wrap the azole compound is selected from the group consisting of polyacrylic acid, a copolymer of polyacrylic acid, polymethacrylic acid and a copolymer of polymethacrylic acid. The copolymers of poly(meth)acrylic acid may be copolymers of (meth)acrylic acid with another (meth)acrylic derivative, e.g. alkyl (meth)acrylate. In one preferred embodiment, the amphiphilic polymer is polyacrylic acid. In another preferred embodiment, the amphiphilic polymer is a copolymer of acrylic acid with butyl acrylate in different proportions (see Table 5).

In yet another preferred embodiment, the nano-particles of the invention comprise inclusion complexes in which the active compound is a taxane and the amphiphilic polymer is gelatin.

As used herein, the term “taxane” refers to compounds containing the twenty carbon taxane core framework represented by the structural formula shown, for example, in U.S. Pat. No. 6,201,140, herein incorporated by reference in its entirety as if fully disclosed herein. The term taxane includes the chemotherapy agents Taxol (generic name: paclitaxel; chemical name: 5β,20-epoxy-1,2a,4,7β,10β,13a-hexahydroxytax-11-en-9-one, 4,10-diacetate 2-benzoate 13-ester with (2R,3S)-N-benzoyl-3-phenylisoserine) and Taxotere (generic name: docetaxel) and semy-synthetic derivatives of taxanes having, for example, an ester or ether substituent at C(7), a hydroxy substituent at C(10), and a range of C(2), C(9), C(14), and side chain substituents, as described for example in the patents U.S. Pat. No. 6,794,523, U.S. Pat. No. 6,780,879, U.S. Pat. No. 6,765,015, U.S. Pat. No. 6,610,860, U.S. Pat. No. 6,552,205 and U.S. Pat. No. 6,201,140, all these patents being herein incorporated by reference in their entirety as if fully disclosed herein.

Taxol, an anticancer drug that now has the generic name “paclitaxel”, and the registered tradename “Taxol®” (Bristol-Myers Squibb Company), is a complex polyoxygenated diterpene originally isolated from the bark of the Pacific yew tree (Taxus brevifolia). It has been approved by the FDA to treat breast, ovarian, and lung cancers as well as AIDS-related Kaposi's sarcoma. Docetaxel (Taxotere-R), a substance that is similar to paclitaxel and also comes from the needles of the yew tree, has been approved by the FDA to treat advanced breast and non-small cell lung cancers that have not responded to other anticancer drugs. Paclitaxel and docetaxel are administered intravenously. Both paclitaxel and docetaxel have side effects that can be serious. Paclitaxel is a white to off-white crystalline powder. This natural compound is highly hydrophobic, insoluble in water. One problem associated with the administration of taxol is its low solubility in most pharmaceutically-acceptable solvents; the formulation used clinically contains Cremophor EL (polyethoxylated castor oil) and ethanol as excipients, which cause serious adverse effects. Thus, in spite of paclitaxel's good clinical efficacy and its recognized as one of the biggest advances in oncology medicine, there is still a growing need to achieve better safety and pharmacokinetic profile of paclitaxel in patients.

U.S. Pat. No. 6,753,006 discloses stable, sterile, nonaqueous formulations containing a sufficient quantity of non-crystalline, cremophor-free paclitaxel to allow systemic administration to a human of a dose in the range of 30-1000 mg/m².

In accordance with the present invention, water-soluble nano-particles are provided comprising inclusion complexes in which paclitaxel in a non-crystalline state, e.g. amorphous state, is wrapped by gelatin and is fixated/stabilized by non-valent interactions with the surrounding gelatin. In preferred embodiments, vitamin B12 and/or polystyrene sulfonic acid are added to the gelatin to increase solubility of paclitaxel.

The aqueous nano-dispersions of the invention can be lyophilized and then mixed with pharmaceutically acceptable carriers to provide stable pharmaceutical composition.

The pharmaceutically acceptable carriers or excipients are adapted to the type of active compound and the type of formulation and can be chosen from standard excipients as well-known in the art, for example, as described in Remington: The Science and Practice of Pharmacy (Formerly Remington's Pharmaceutical Sciences) 19th ed., 1995.

Thus, in another aspect, the present invention provides stable pharmaceutical compositions comprising pharmaceutically acceptable carriers and a nano-dispersion of the invention. The compositions are preferably for oral administration, and may be in liquid or solid form. In one preferred embodiment, tablets are provided, as exemplified herein for azithromycin.

In one preferred embodiment, the invention relates to stable pharmaceutical compositions for treatment of bacterial infections comprising a nano-dispersion of water-soluble nano-particles comprising an inclusion complex wherein the active compound is a macrolide antibiotic selected from the group consisting of erythromycin, clarithromycin and azithromycin and the amphiphilic polymer is a polysaccharide. These compositions can be useful for any bacterial infection treatable by said macrolide antibiotics and, particularly, for respiratory infections.

In another preferred embodiment, the invention relates to stable pharmaceutical compositions for treatment of dementia and Alzheimer's disease comprising a nano-dispersion of water-soluble nano-particles comprising an inclusion complex wherein the active compound is donepezil hydrochloride and the amphiphilic polymer is a polysaccharide.

In a further preferred embodiment, the invention relates to stable pharmaceutical composition for treatment of fungal infections comprising a nano-dispersion of water-soluble nano-particles comprising an inclusion complex wherein the active compound is an azole fungicide and the amphiphilic polymer is selected from the group consisting of a polysaccharide, polyacrylic acid, a copolymer of polyacrylic acid, polymethacrylic acid and a copolymer of polymethacrylic acid. In a more preferred embodiment, the azole fungicide is itraconazole and the amphiphilic polymer is selected from the group consisting of polyacrylic acid, a copolymer of acrylic acid with butyl acrylate, chitosan, and starch that has been modified by one or more of the following treatments: acid hydrolysis, reaction with polyethylene glycol or hydrogen peroxide, or thermal treatment.

In yet another preferred embodiment, the invention relates to stable pharmaceutical compositions for treatment of estrogen-responsive breast tumors comprising a nano-dispersion of water-soluble nano-particles comprising an inclusion complex wherein the active compound is a nonsteroidal antiestrogen azole selected from the group consisting of letrozole, anastrozole, vorozole and fadrozole.

In still a further preferred embodiment, the invention relates to stable pharmaceutical compositions for treatment of cancer comprising a nano-dispersion of water-soluble nano-particles comprising an inclusion complex wherein the active compound is a taxane, most preferably paclitaxel, and the amphiphilic polymer is gelatin.

The invention will now be illustrated by the following non-limiting examples.

### EXAMPLES

### Example 1

### General Procedure for Production of the Mano-Particles Comprising Inclusion Complexes

For the preparation of the nano-particles of the invention, the following general procedure is carried out:
(i) preparation of a molecular solution of the amphiphilic polymer in water;
(ii) preparation of a molecular solution of the active compound in an organic solvent;
(iii) dripping the cold solution of the active compound (ii) into the polymer solution (i) heated at a temperature 5-10° C. above the boiling point of the organic solvent of (ii), under constant mixing; and
(iv) evaporation of the organic solvent thus obtaining the desired nano-dispersion of nano-particles comprising the inclusion complexes of the active compound entrapped within the amphiphilic polymer.

### Example 2

### Preparation of Modified Starch

For use in the invention, it is desirable to use starch with a large proportion of linear chains, i.e. starch with high contents of amylose, the constituent of starch in which anhydroglucose units are linked by a-D-1,4 glucosidic bonds to form linear chains, and low contents of amylopectin, a constituent of starch having a polymeric, branched structure. The levels of amylose and amylopectin and their molecular weight vary between different starch types.

To improve its characteristics for use in the invention, starch, e.g. corn or potato starch, can be modified, for example by increasing its hydrophilicity by acid hydrolysis and/or by reaction with an agent, e.g. polyethylene glycol (PEG) and or hydrogen peroxide. In addition, starch can be subjected to thermal treatment, for example at 160-180° C., for about 30-60 min, to reduce the amount of branching (hereinafter designated “thermodestructed starch”).

For modification, varying amounts of potato starch and distilled water were put into a reaction vessel (C p.st=concentration of potato starch, X1 in Table 1) and citric acid was added under mixing until the desired pH (range 2-5) was attained (X2, Table 1). The obtained suspension was heated from room temperature to 70-95° C., for approximately 10-20 minutes with continuous mixing until a homogeneous opaque mass was obtained (hydrolyzed starch). The obtained mass was exposed to 160-180° C. in an autoclave for time X3 (min). Under these conditions, the network structures of starch are partially or completely transformed to linear weakly branched macromolecules which dissolve in water. The mass was cooled below 100° C. (thermodestructed starch).

To some of the samples, PEG-400 was added (amount X4, % in relation to starch, Table 1), the obtained mixture was heated at 160-180° C. in an autoclave for time X5 (Table 1), and thereafter cooled below 100° C. (PEG-modified thermodestructed starch). Turbidity (in FTU, Formazin Turbidity Unit) and viscosity (molecular weight, MW) of the solution were measured. The results are shown in Table 1. The solution appropriate for further use should be transparent or opalescent, and should have preferably a turbidity within the range 20-40 FTU. Furthermore, the molecular weight (MW) of modified starch is calculated according to intrinsic viscosity measurements. Acceptable MW (as reflected by the intrinsic viscosity) values are up to approximately 100,000 and depend on the active compound to be complexed.

TABLE 1
Characteristics of modified potato starch

| C p.st., % | PH | T1 min | PEG-400, | T2 min | Turbidity | MW |
|---|---|---|---|---|---|---|
| X1 | X2 | X3 | X4 % | X5 | FTU | (Visc.) |
| 4 | 5 | 60 | 0 | 0 | 26 | 9785 |
| 4 | 5 | 60 | 0 | 0 | 32 | 39212 |
| 4 | 5 | 60 | 0 | 0 | 32 | 71623 |
| 4 | 5 | 60 | 0 | 0 | 30 | 74900 |
| 4 | 5 | 60 | 0 | 0 | 36 | 98236 |
| 4 | 5 | 60 | 0 | 0 | 31 | 91872 |
| 4 | 5 | 60 | 1 | 0 | 33 | 7082 |
| 4 | 5 | 60 | 1 | 0 | 43 | 74747 |
| 8 | 2 | 150 | 0 | 0 | 11 | 5095 |
| 8 | 3 | 30 | 2 | 30 | 146 | 21680 |
| 8 | 2 | 150 | 4 | 60 | 2 | 8500 |

C p. st = concentration of potato starch

### Example 3

### Preparation of Nano-Particles Comprising Inclusion Complexes of clarithromycin Wrapped in Modified Starch

For the preparation of the amphiphilic polymer, potato starch of molecular mass (5−10)×10⁴ was dissolved in distilled water, initially heated at 160-180° C., and modified by PEG-400 as described in Example 2, using starch: PEG-400 ratio ranges between 2:1 and 4:1, solution pH (6.5 or below) adjusted with citric acid, temperature 160-180° C., and time of modification 60-180 min. A solution of clarithromycin in methyl acetate or dichloromethane was prepared.

The aqueous solution of the modified starch was put in a reaction vessel and heated up to 60° C. while mixing with a homogenizer at speed of 10,000 and up rev/min. After the temperature of the starch solution reached 60° C., the clarithromycin solution was added thereto at a rate of about 1 ml/sec. The homogenizer speed was also at least 10,000 rev/min. Clarithromycin interacted with the modified starch to create nano-particles, and the organic solvent was evaporated and condensed in a direct condenser. After all the clarithromycin had interacted with the polymer and had solubilized as a clarithromycin-starch inclusion complex, the residual organic solvent was vacuum evaporated with continuous mixing, and the aqueous solution of the nano-particles comprising chlarithromycin-starch inclusion complexes was cooled to 30-35° C.

The turbidity and viscosity of the cooled aqueous solution of the nano-particles were measured for predetermined storage periods in order to assess the dispersion stability. The turbidity values for nano-dispersions of several clarithromycin-starch inclusion complexes are shown in Table 2. A stable nano-dispersion has a turbidity that remains unchanged over time. The presence of a crystalline phase, and particles sizes of the complex were determined.

TABLE 2
Complexes of Clarithromycin wrapped within starch

| | | Store | Turbidity |
|---|---|---|---|
| Product | | Time | (FTU) at room |
| number | Solution components | (days) | temperature |
| 39 | Hydrolyzed potato starch - 5%, | 0 | 29 |
| | Clarithromycin - 1%, | 4 | 27 |
| | pH 5.0 | 10 | 35 |
| | | 20 | 28 |
| | | 26 | 30 |
| 37 | Hydrolyzed potato starch - 4%, | 0 | 38 |
| | Clarithromycin - 2%, | 5 | 40 |
| | pH 4.5 | 21 | 36 |
| | | 27 | 43 |
| 40 | Hydrolyzed potato starch - 4%, | 0 | 36 |
| | Clarithromycin - 2%, | 1 | 36 |
| | pH 5.5 | 7 | 37 |
| | | 17 | 36 |
| 42 | Hydrolyzed modified (50% PEG) | 0 | 40 |
| | potato starch - 6%, | 1 | 40 |
| | clarithromycin - 1%, | 6 | 39 |
| | pH 4.5 | 16 | 41 |
| | | 22 | 40 |
| 34 | Hydrolyzed modified (25% PEG) | 0 | 21 |
| | potato starch - 3.75%, | 10 | 20 |
| | clarithromycin - 1%, | 16 | 19 |
| | pH 4.5 | 26 | 20 |
| 36 | Hydrolyzed modified (50% PEG) | 0 | 46 |
| | potato starch - 6%, | 15 | 48 |
| | clarithromycin - 1.5%, | 21 | 47 |
| | pH 5.0 | 30 | 49 |
| 38 | Hydrolyzed modified (30% PEG) | 0 | 26 |
| | potato starch - 5.2%, | 4 | 28 |
| | clarithromycin - 1.7%, | 10 | 27 |
| | pH 5.5 | 20 | 26 |
| 43 | Hydrolyzed modified (50% PEG) | 0 | 38 |
| | potato starch - 12%, | 1 | 37 |
| | clarithromycin - 2.5%, | 5 | 39 |
| | pH 6.5 | 15 | 37 |
| | | 25 | 38 |
| 46 | Hydrolyzed modified (50% PEG) | 0 | 36 |
| | potato starch - 6%, | 1 | 40 |
| | clarithromycin - 2.5%, | 3 | 44 |
| | pH 5.0 | 9 | 48 |
| | | 10 | 50 |
| | | 17 | 47 |
| 47 | Hydrolyzed potato starch - 3.75%, | 0 | 32 |
| | clarithromycin - 1.5%, | 1 | 31 |
| | pH 4.5 | 6 | 35 |
| | | 14 | 32 |
| 48 | Hydrolyzed potato starch - 3% | 0 | 25 |
| | clarithromycin - 1.5%, | 1 | 25 |
| | pH 5.0 | 2 | 26 |
| | | 8 | 25 |
| 50 | Hydrolyzed potato starch - 8%, | 0 | 70 |
| | clarithromycin - 1%, | 1 | 77 |
| | pH 5.0 | 4 | 79 |
| | | 6 | 78 |
| 51 | Hydrolyzed modified (25% PEG) | 0 | 64 |
| | polysacharide - 5%, | 1 | 62 |
| | clarithromycin - 3%, | 2 | 65 |
| | pH 5.0 | 3 | 61 |
| | | 10 | 64 |

Stable Turbidity = stable nano-dispersion.

### Example 4

### In vitro Microbiological Activity of Nano-Particles of clarithromycin-Modified Starch Inclusion Complexes

The microbiological activity of various concentrations of water-soluble complexes of clarithromycin prepared in Example 3 was tested on the bacterium Micrococcus luteus, which is sensitive to macrolide antibiotics, and compared to uncomplexed commercial clarithromycin using well-known agar-filled petri-dish tests. Small filter paper cut discs were impregnated with two different solution concentrations (100 or 1,000 μ/ml) of the tested antibiotics. Diameters of the zones pf bacteriostatic activity were measured versus time. Concentrations were varied significantly for both control (commercial clarithromycin) and complexed clarithromycin and observed until 216 hours post-application. The results are illustrated in FIG. 1 and demonstrate that the complexed clarithromycin show the same microbiological activity as commercial clarithromycin while using 1/10 of the amount (concentration). Furthermore, for identical concentrations of drug, the uncomplexed clarithromycin antibacterial activity ceased at approximately 48 hours, while that of the complexed clarithromycin continued significantly until approximately 216 hours. It was also observed that the difference in microbiological activity for complexed clarithromycin having concentration differences of an order of magnitude between them is vastly greater than the corresponding differences noted with uncomplexed clarithromycin.

### Example 5

### In vivo Studies with clarithromycin-Modified Starch Complexes

Rats (male Sprague-Dawley (Harlan), n=5 in each group) were orally administered (gavage) 150 mg/kg water-soluble clarithromycin nano-particle complexes of Example 3 or uncomplexed commercial clarithromycin (200 mg/kg). At various time intervals, blood samples were collected via a jugular catheter and the plasma concentrations of the complexed clarithromycin (Soluclari) and of the commercial drug were determined.

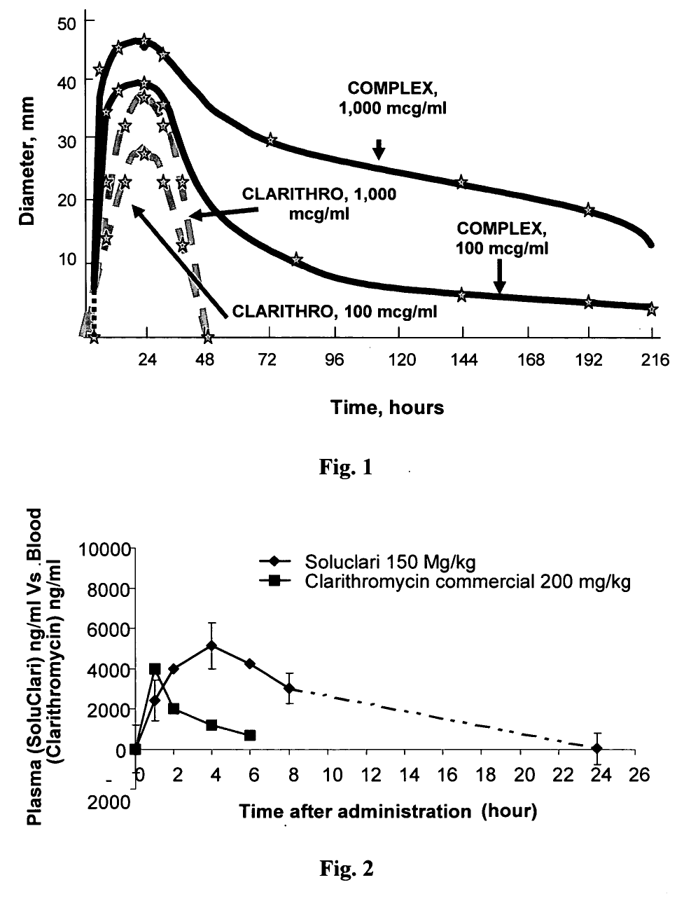

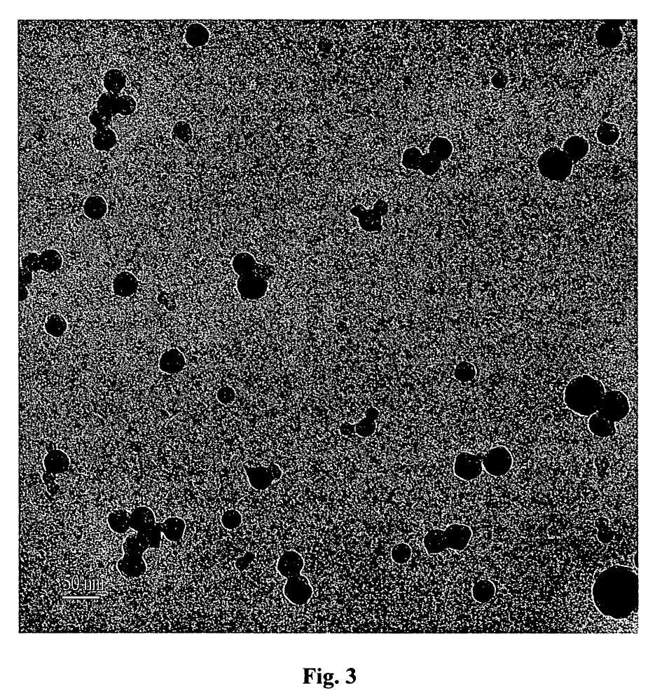

The results are shown in FIG. 2. Values at time 0 were the control baseline for each animal. Following oral administration of clarithromycin in nano-particle complex, it was determined that the drug reached its maximum plasma value 4 hours following administration. The first absorption phase was rapid—up to 1 hour and continued until maximum at 4 hours. The clearance was significantly slow in comparison to published data with the commercial clarithromycin. The circulating half-life was in the range of 2 hours. The Area Under the Curve (AUC_{0-24 hours}) of the clarithromycin complex in accordance with the present invention was significantly higher (54.2 μg×h/ml) in comparison to published data with the same dose of the commercial clarithromycin in rats (AUC_{0-24 hours}=32.54 (μg×h/ml) with same oral dose of 150 mg/kg. This indicates that the nano-particles comprising complexed clarithromycin in accordance with the present invention exhibit either enhanced bioavailability or intestinal slow release following oral administration.

### Example 6

### Physical Measurements and Characteristics of clarithromycin and erythromycin in Nano-Particle Complexes with Starch

(i) Particle Size and Distribution of the Inclusion Complexes

Complexes of erythromycin with modified starch were prepared from erythromycin dissolved in organic solvent as described for clarithromycin in Example 3 above. In both cases, the technology of the present invention allowed the creation of drug-polymer nano-dispersions with controllable nano-particle sizes, ranging from single nanometers up to 1000 nm, with a highly uniform size distribution. A complex of clarithromycin prepared according to the method of the present invention showed identical dispersion spectra after 5 weeks time.

Size measurements of the inclusion complexes were performed using ALV-Particle Sizer (ALV-Laser GmbH, Langen, Germany), which has a resolution of 3-3000 nm. ALV is a dynamic light scattering technique used to estimate the mean particle size. Experiments are conducted with a laser-powered Noninvasive Back Scattering=High Performance Particle Sizer (ALV-NIBS/HPPS).

A comparison of particle size measurement by light diffraction (ALV) and scanning electron microscopy (SEM) was carried out with clarithromycin-starch inclusion complexes nano-particles. FIG. 3 is an SEM micrograph of nano-particles comprising the clarithromycin-hydrolyzed potato starch complex (#IC-76, Table 3 hereinbelow) showing that the particles have a size of approximately 100 nm. This is a value significantly smaller than the ALV measurement shown in Table 3 (407 nm). The difference may be attributed to freeze-drying of the sample prior to SEM analysis. Based on this and previous evidence, it appears that freeze-drying may remove the hydrous layer that is measured in the ALV analysis.

(ii) Solubility

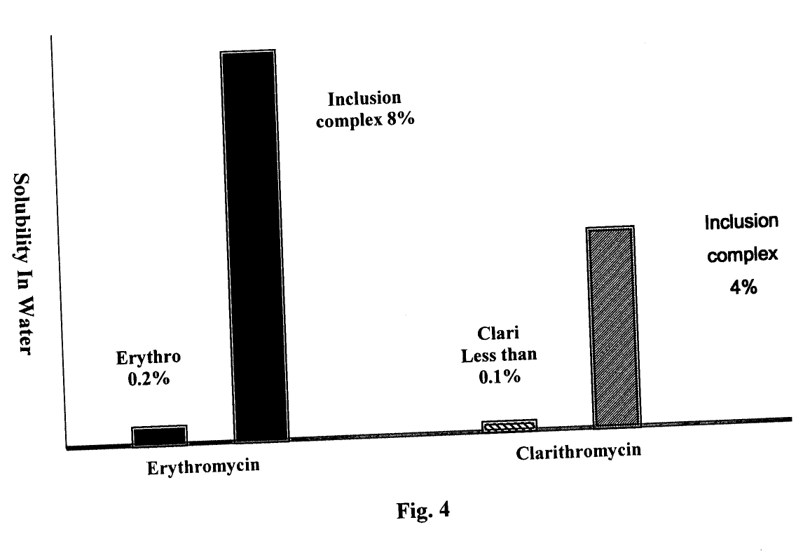

Erythromycin, an antibiotic practically insoluble in water, was complexed with modified starch into thermodynamically stable nano-dispersions, with controllable size distribution of the particles in the dispersed phase. The resulting nano-dispersions had 8% (w/v) active drug, which is 40 times higher than the solubility of the original drug in water (0.2%). Moreover, drug particles with a highly uniform size of complexes (over 95%) were achieved. The erythromycin was released from the inclusion complex in sufficient concentration under physiological-like conditions. No existing technologies of solubilization were used, e.g. surfactants, liposome, capsulation, etc. A comparison of the solubility of commercial erythromycin and clarithromycin and as nano-particles of the inclusion complexes with modified starch is illustrated in FIG. 4.

(iii) Stability of the Nano-Particles

Observations were made of transparent aqueous solution of inclusion complexes for non-occurrence of phase separation and maintenance of particle size and size distribution. The following observations and results were obtained:
(a) Over 75 days, the tests of the aqueous solutions of 8% erythromycin showed no phase separation and showed maintenance of particle size and size distribution.
(b) The stability of the nano-particles of clarithromycin-starch complexes was observed for 12 weeks at room temperature and 4 weeks at 35° C. and were found to be stable.
(c) Following freeze-drying and subsequent rehydration of complexed clarithromycin-starch, the particle size of the drug-polymer complexes is retained and the amorphicity of clarithromycin is also retained. For more than 30 days there was no aggregation and the nano-dispersion was stable.

(iv) X-Ray Diffraction Results and Characterizations

The following X-ray method and equipment were used: X-ray diffraction patterns were collected with CuKa radiation on the Scintag theta-theta Powder Diffractometer equipped with liquid nitrogen-cooled, solid-state Ge detector.

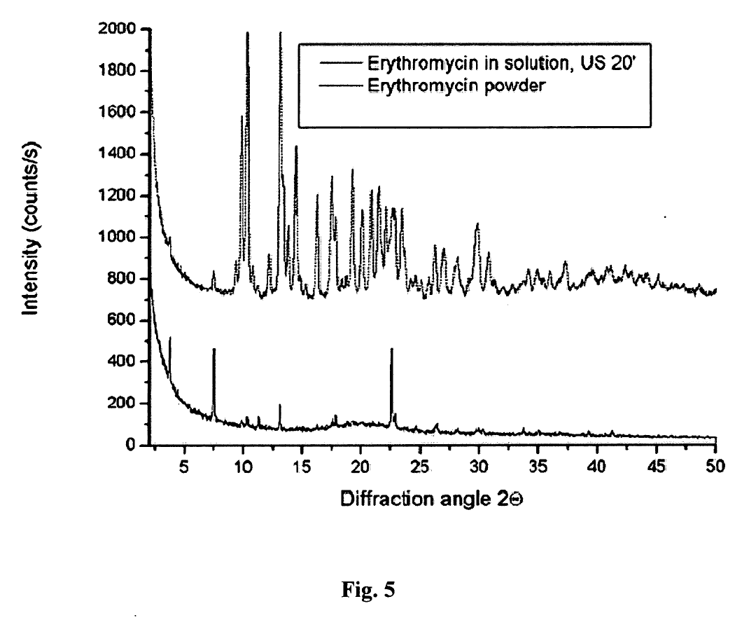

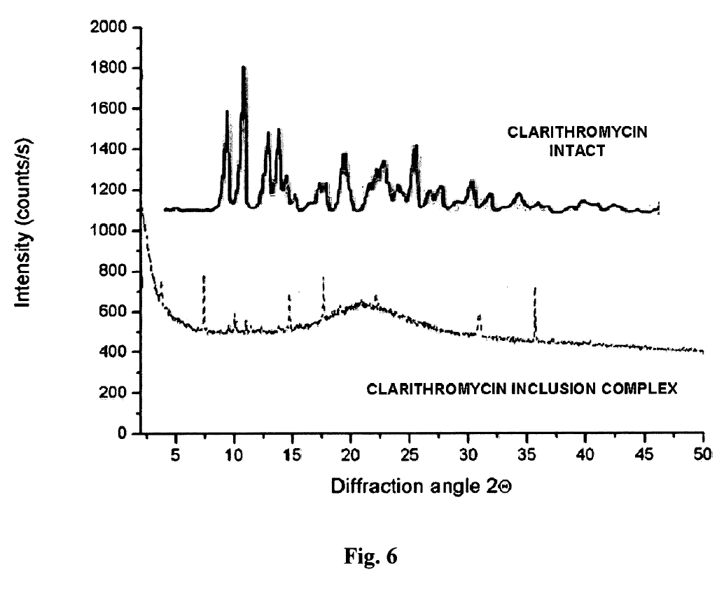

Powder X-ray diffraction measurements showed that preparation of nano-dispersions of crystalline drug erythromycin resulted in its conversion into an amorphous form material. FIG. 5 and FIG. 6 illustrate the X-ray diffraction comparisons of intact erythromycin and intact clarithromycin compared with the inclusion complexes of erythromycin-starch and clarithromycin-starch, respectively.

The comparison of known spectra of erythromycin (FIG. 5) and clarithromycin (FIG. 6) with the inclusion complexes in accordance with the present invention were conducted. The known spectrum of erythromycin as a dry powder depicted in FIG. 5 shows a well-defined crystalline pattern. In comparison, the spectrum of the erythromycin inclusion complex, depicted in FIG. 5, demonstrates that the majority of peaks derived from crystalline erythromycin are not present, and the few remaining peaks have been drastically reduced in height. This spectrum is undoubtedly related to that of the known erythromycin, however it is indicative that another “form” is now present after complexation.

When observing the average scattering angles in the spectra of both complexed erythromycin and clarithromycin one can clearly see that certain peaks have been “flattened” showing widened virtually base line peaks. This phenomenon is indicative of an amorphous state.

These results show that complexation of erythromycin and clarithromycin using the technology of the present invention reduces crystallinity of the uncomplexed drugs, as the crystal lattices are unable to form, due to fixation of the drugs within the inclusion polymer on the basis of Van der Waals and hydrogen bonds. It is known that the amorphous state is preferred for drug delivery as it may indeed enhance bioavailability.

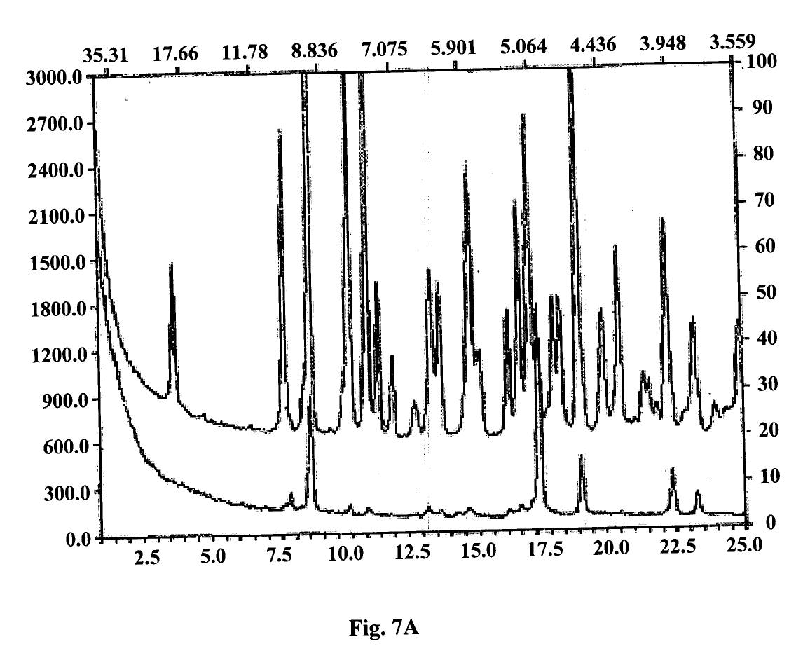

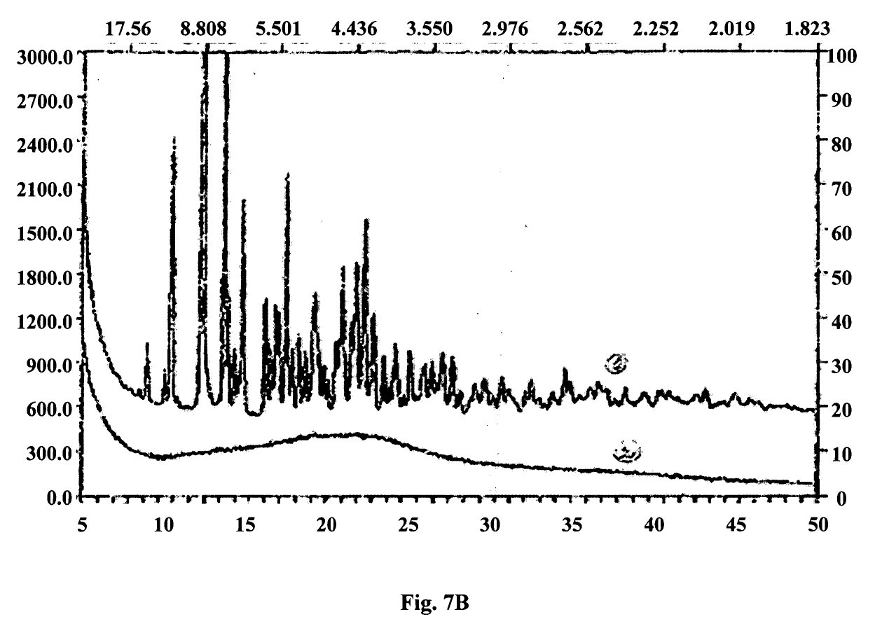

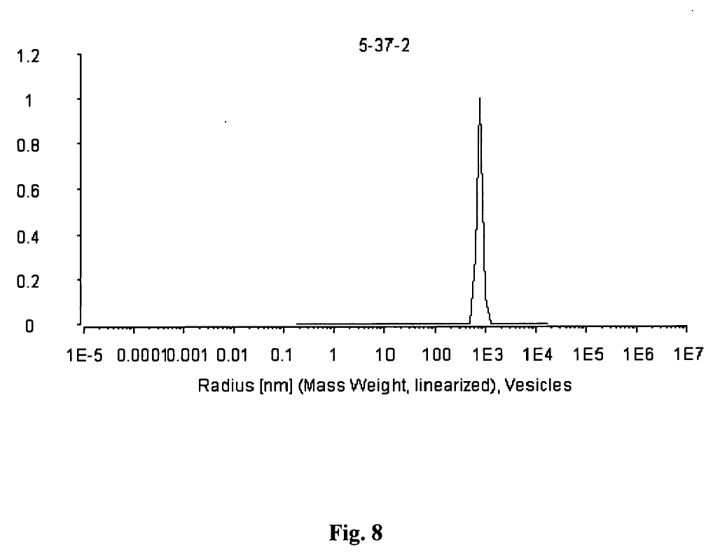

The X-ray spectrum of FIG. 7A depicts a 6-month old clarithromycin complexed sample (bottom trace) compared to the commercially available clarithromycin (upper trace). This specific complexed sample is identical to that appearing in FIG. 6 and in the microbiological tests discussed in Example 4. This validates the technological ability to prepare uniquely complexed drug conjugates in accordance with the present invention that demonstrate significantly stabilized amorphous states. FIG. 7B illustrates X-ray spectra of 10-month old azithromycin-chitosan inclusion complex sample (bottom trace) compared to the commercially available azithromycin (upper trace) (see Example 10 hereinafter).

The present inventors believe that such stabilization of amorphous or partially amorphous drug states within the inclusion complex may well increase the chances of greater bioavailability as has been documented in the literature. Taken together with other parameters attained using the process and apparatus of the present invention, such as very accurate size control, the process lends itself easily to significantly increased bioavailabilities.

### Example 7

### Controlled Release of erythromycin from the erythromycin-starch Inclusion Complex via Dialysis

The preparation of nano-dispersions of a drug as an inclusion complex according to the present invention represents a new avenue to achieve controlled release systems that deliver the drug at a specific rate and pattern. To examine the experimental controlled release pattern of erythromycin from the inclusion complex, a dialysis method was performed mimicking physiological conditions. In this method, the drug-polymer nano-dispersions were placed within a dialysis membrane bag. Such a membrane allows the diffusion of only molecules and ions of sizes less than 3000 Da, while maintaining the nano-dispersions. Dialysis was performed for 24 hours at room temperature with constant stirring. Samples from the external buffer were taken periodically for the analysis of drug release. The concentration of erythromycin released from the inclusion complex with starch was detected by measuring the O.D. (optical density). After 24 hours of incubation, the concentration of erythromycin in the external fluid was 25% of the initial concentration of erythromycin in the inclusion complex (initial concentration is 4 mg/ml (8% w/v)). The released concentration also reflects the maximum solubility of erythromycin in a serum-modeled solution. Thus, this result indicates that the nano-dispersion has a capability to sustain the release of erythromycin.

### Example 8

### Physical Characteristics of Further clarithromycin-polymer Inclusion Complexes

Further inclusion complexes of clarithromycin hydrophilic inclusion complexes were prepared according to the method described in Example 1, in which clarithromycin was dissolved in methyl acetate or dichloromethane and the polymers were hydrolyzed potato starch, alginate, chitosan or polyvinyl alcohol (PVA).

Table 3 below shows the properties of various such complexes. Shown in Table 3 are complex designation (Exp., first column), polymer name and concentration (%), drug concentration, pH, and physico-chemical analysis of the various complexes nano-particles including ALV-size and size distribution (nm), HPLC (concentration and thus solubility) and, in some cases, powder X-ray analyses for the determination of crystalline phase. Size measurements of the complexes performed using ALV technique and powder X-ray analyses were carried out as described in Example 6 above.

FIG. 8 illustrates the size distribution of of nano-particles comprising the clarithromycin hydrophilic inclusion complex within 1% chitosan (# 10-134 in Table 3) having a size of approximately 838 nm.

TABLE 3
Properties of Clarithromycin Hydrophilic Inclusion Complexes

| | | | | HPLC | | Size | |
|---|---|---|---|---|---|---|---|
| | Polymer | Drug | | Quantity | % of | Distribution | |
| Exp. | (name/%) | (mg/ml) | pH | (ml) | Initial | nm | X-Ray |
| IC-76 | Hydrolyzed | 2 | 5 | ND | ND | 407 | ND |
| | potato starch | | | | | | |
| | 4% dil to | | | | | | |
| | 2% | | | | | | |
| IC-98 | Hydrolyzed | 10 | 5 | 5 | 93.9 | ND | Amorphous |
| (75) | potato starch | | | | | | |
| | 4% dil to 2% | | | | | | |
| IC-133 | 2% Alginate | 10 | 5.5 | 20 | 44.3 | 530 | Amorphous |
| | Kelton LV | | | | | | |
| IC-135 | 1% Chitosan | 10 | 4-6 | 5 | 84.5 | 165 | Amorphous |
| | Fluka | | | | | | |
| | 50494 | | | | | | |
| 1Cl-IZ- | 2% PVA | 10 | 6 | 35 | 76.9 | 1600 | Crystalline |
| 10-34 | | | | | | | |
| 1Cl-IZ- | 1% Chitosan | 10 | 4 | | 91.3 | 321 | ND |
| 135/1- | Fluka | | | | | | |
| 10-112 | 50494 | | | | | | |
| 1Cl-IZ- | 1% Chitosan | 10 | 6 | | 99.6 | 660 | ND |
| 135/1- | Fluka | | | | | | |
| 10-112 | 50494 | | | | | | |
| 1Cl-IZ- | 1% Chitosan | 10 | 5 | No | 55.4 | 838 | ND |
| 135/2- | Sigma | | | | | | |
| 10-134 | C3646 | | | | | | |

Dil = diluted;
LV = low viscosity;
HPLC = High Performance Liquid Chromatography;
ND = not done

As shown in Table 3, nano-particles (size below 1000 nm) could be prepared using polymers such as hydrolyzed potato starch, alginate, and chitosan from different sources, but with PVA the particles had a size of 1600 nm and the particles were crystalline and not amorphous, indicating that apparently PVA is not useful for preparing macrolide-containing nano-particles.

The results in Table 3 show that, when the macrolide antibiotic clarithromycin, which is a poorly soluble hydrophobic compound, is surrounded by an amphiphilic polymer, the resulting inclusion complex is hydrophilic. Using the matching technique described in the instant application, clarithromycin was rendered hydrophilic when surrounded by various polymers which meet the matching parameters; such as alginate, PVA and chitosan. The results show that 2% alginate combined with 10 mg/ml of clarithromycin at pH 5.5 resulted in nanoparticles with a ALV size distribution analysis of 530 nm; 2% PVA combined with clarithromycin at pH 6 resulted in nanoparticles with a ALV of 1600 nm; 1% chitosan (Fluka) combined with clarithromycin at pH 4-6 resulted in a ALV of 165 nm; 1% chitosan (Fluka) combined with clarithromycin at pH 4 resulted in an ALV of 321 nm; 1% chitosan (Fluka) combined with clarithromycin at pH 6 resulted in an ALV of 660 nm; and 1% chitosan (Sigma) combined with clarithromycin at pH 5 resulted in an ALV of 838 nm. These results show that using the teachings of the specification (lipophilic/amphiphilic polymer matching technique), a poorly soluble hydrophobic antibiotic (clarithromycin) can be surrounded by various amphiphilic polymers (i.e. alginate, PVA and chitosan) to render the resulting inclusion complex hydrophilic in water.

It was also found that identical dispersion spectra of the clarithromycin complexes are obtained immediately and 5 weeks after preparation. As recited above, FIG. 3 is a SEM micrograph that illustrates the consistent spherical nano-particles of the clarithromycin-hydrolyzed starch inclusion complex IC-76 (Table 3).

### Example 9

### Controlled Release of clarithromycin from clarithromycin-polymer Inclusion Complexes via Dialysis

This experiment was carried out as in Example 7, using a cellulose dialysis membrane of molecular weight cut-off of 3500 D (SnakeSkin™ Dialysis Tubing, Pierce Chemical Co., Product #68035).

Three formulations of clarithromycin were tested:
1. Commercial clarithromycin dissolved in water (10 mg/ml), pH 4.
2. Clarithromycin complex in 2% PVA of Table 3, with initial calculated concentration 10 mg/ml, pH=6.0 (S-Clari#34)
3. Clarithromycin complex in 1% chitosan (IC-135 of Table 3) with initial calculated concentration 10 mg/ml, pH=6.5 (S-Clari#135)

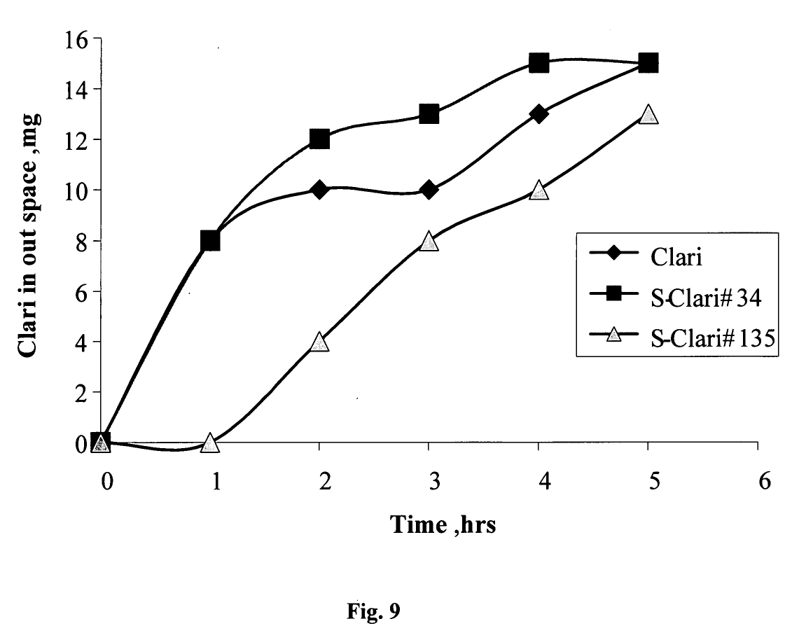

Each formulation (2 ml) was put in a dialysis sac placed in a glass jar with 100 ml water, pH=4 (titration with citrate 20%). Dialysis was performed for up to 6 hours under constant stirring at 23±2° C. Samples (1 ml) of external buffer were taken each hour during 5 hours of incubation for the analysis of drug release. Volume of exterior fluid was constantly 100 ml. The concentration of clarithromycin in external (out of sac) and internal (in the sac) fluids and tested samples were determined by HPLC. The results, depicted in FIG. 9, show that release of clarithromycin from the PVA complex (S-Clari#34, squares) is faster than that of the commercial formulation (Clari, losanges), while in contrast, release of clarithromycin from the chitosan complexes (S-Clari#135, triangles) is significantly slower than that of the commercial formulation. This indicates that the nano-dispersion with chitosan has a capability to sustain the release of clarithromycin and is more suitable for the preparation of the inclusion complex with the macrolide. As shown in Table 3, the complex with PVA had a size of 1600 nm, not within the nano-range, thus it did not have sustained release.

### Example 10

### Physical Measurements and Characteristics of Various Azithromycin Hydrophilic Inclusion Complexes

Inclusion complexes of another macrolide antibiotic, azithromycin, were prepared according to the method described in Example 1, in which azithromycin was dissolved in methyl acetate or dichloromethane and the polymers were alginate, manucol ester B (an alginate derivative), chitosan or PVA.

Table 4 below shows the properties of various such complexes. Shown in Table 4 are complex designation (Exp., first column), polymer name and concentration (%), drug concentration, pH, and physico-chemical analysis of the various complexes nano-particles including ALV-size and size distribution (nm) and HPLC (concentration and thus solubility). Size measurements of the complexes performed using ALV technique were carried out as described in Example 6 above.

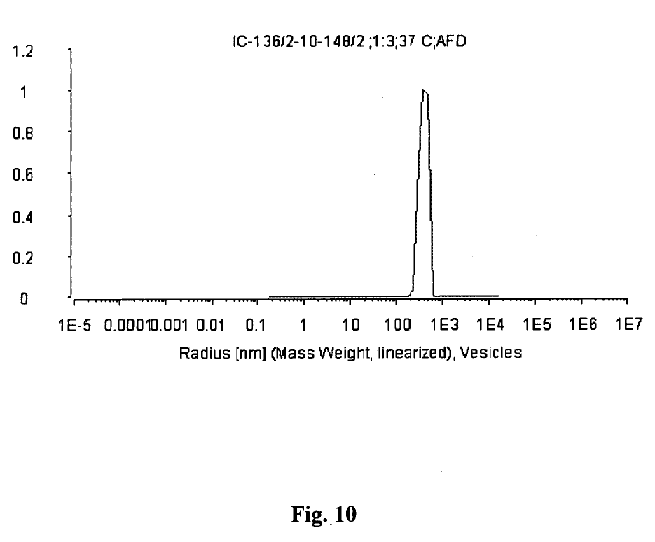

FIG. 10 illustrates the size distribution of nano-particles comprising the azithromycin hydrophilic inclusion complex within 1% chitosan (# 10-148/2 in Table 4) having a size of approximately 362 nm. Furthermore, azithromycin in these particles was found to amorphous, and as shown in the lower trace of FIG. 7B, the amorphocity was found to be stable for at least ten months.

TABLE 4
Properties of Azithromycin Hydrophilic Inclusion Complexes

| | | | HPLC | Particle |
|---|---|---|---|---|
| | Polymer | Drug | % of | Size |
| Exp. | (name/%) | (mg/ml) | Initial | nm |
| 2AZ-IZ-10-32 | 2% PVA | 10 | 99 | 5 |
| 2AZ-IZ-10-36 | 4% Manucol | 10 | 90.4 | 330 |
| | (Alginate) Ester B | | | |
| 2AZ-IZ-10-42 | 1% PVA | 10 | 94 | 5 |
| AZ-IC-131/1-IZ-10-145 | 2% Alginate | 20 | 82.6 | 1600 |
| | (Kelton) LV | | | |
| AZ-IC-10-42/1-IZ-10- | 1% PVA | 10 | 99.32 | 350 |
| 146 | | | | |
| AZ-IC-134/1-IZ-10-147 | 2% Alginate | 10 | 98.06 | 1060 |
| | (Kelton) LV | | | |
| AZ-IC 136/2-IZ-10-148 | 1% Chitosan | 10 | 97.16 | 510 |
| | (Sigma) C3646 | | | |
| AZ-IC 136/3-IZ-28-1 | 1% Chitosan | 10 | 95.36 | 752 |
| | (Sigma) C3646 | | | |
| AZ-IC 136/2-10-148/2 | 1% Chitosan | 10 | 97 | 362 |
| | (Sigma) C3646 | | | |

HPLC = High Performance Liquid Chromatography assay

The results in Table 4 show that when the macrolide antibiotic azithromycin, which is a poorly soluble hydrophobic compound, is surrounded by an amphiphilic polymer, the resulting inclusion complex is hydrophilic. Using the matching technique described in the instant application, azithromycin is rendered hydrophilic when surrounded by various polymers which meet the matching parameters such as alginate, PVA, Manucol Ester B, and chitosan. The results show that 2% PVA combined with 10 mg/ml of Azithromycin resulted in a nanoparticle size distribution analysis of ALV of 5 nm; 4% Manucol Ester B combined with azithromycin resulted in an ALV of 330 nm; 1% PVA combined with azithromycin resulted in an ALV of 5 nm and, in another formulation process, 350 nm; 2% alginate combined with azithromycin resulted in an ALV of 1600 nm and, in another formulation process, 1060 nm; 1% Chitosan (Sigma) combined with azithromycin resulted in a ALV of 510 nm and, in other formulation processes, 752 nm and 362 nm. These results are consistent with the results above with clarithromycin and show that using the teachings of the specification (lipophilic/amphiphilic polymer matching technique), a poorly soluble hydrophobic antibiotic (azithromycin) can be surrounded by various amphiphilic polymers (i.e. alginate, PVA, Manucol Ester B, and chitosan) to render the resulting inclusion complex hydrophilic in water.

### Example 11

### Physical Measurements and Characteristics of Various Itraconazole Hydrophilic Inclusion Complexes

Inclusion complexes of the azole fungicide itraconazole were prepared according to the method described in Example 1, in which itraconazole was dissolved in methyl acetate or dichloromethane and the polymers were hydrolyzed potato starch, thermodestructed potato starch, alginate, chitosan, polyacrylic acid and a copolymer acrylic acid-butyl acrylate.

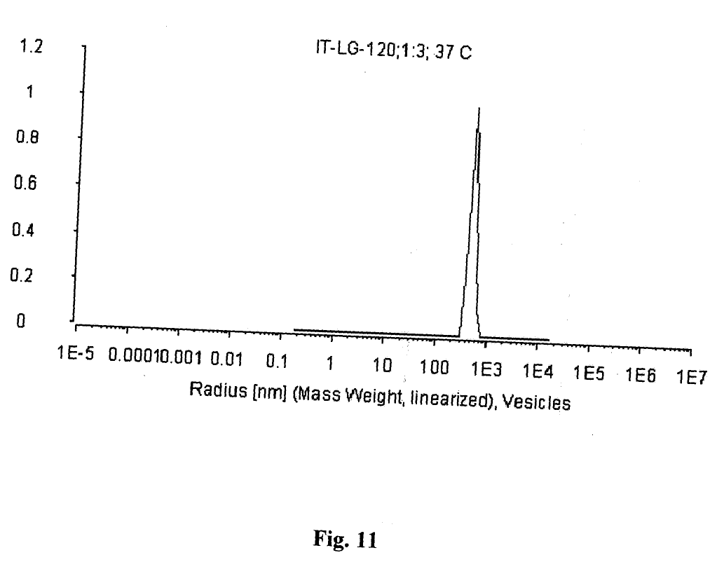

Table 5 below shows the properties of various such itraconazole hydrophilic inclusion complexes. FIG. 11 illustrates the size distribution of nano-particles comprising itraconazole hydrophilic inclusion complexes within thermodestructed starch (# 23-120) having a size of approximately 414 nm.

TABLE 5
Properties of itraconazole hydrophilic inclusion complexes

| | | | HPLC | Particle |
|---|---|---|---|---|
| | | Drug | % of | Size |
| Exp | Polymer (name/%) | (mg/ml) | Initial | nm |
| 07IT-IZ-10-91 | 5% Hydrolyzed potato starch + 1% | 2 | 83.8 | ND |
| | H₂O₂ | | | |
| 07IT-IZ-10-105 | 4% Hydrolyzed potato starch + 1% | 5 | 86.28 | ND |
| | PEG | | | |
| 07IT-IZ-10-140 | 1% Chitosan (Sigma) C3646 | 5 | | ND |
| 7IT-LG-23-104 | 5% Hydrolyzed potato starch + 1.25% | 5 | 101.8 | 382 |
| | H₂O₂ + 1.25% PEG | | | |
| 7IT-LG-23-112 | 5% Thermodestructed potato | 5 | 99.5 | 640 |
| | starch + 0.625% | | | |
| | H₂O₂ + 1.25% PEG | | | |
| 7IT-LG-23-120 | 5% Thermodestructed starch + 1% | 5 | 100 | 414 |
| | H₂O₂ + 2% PEG | | | |
| 7IT-LG-23-113 | 5% Thermodestructed starch + 1% | 5 | 90.41 | 793 |
| | H₂O₂ + 1% PEG | | | |
| IC-131 | 2% Alginate (Kelton) LV | 20 | 101 | 180 |
| IC-134 | 2% Alginate (Kelton) LV | 10 | 95 | 1250 |
| IC-136 | 1% Chitosan (Fluka) 50494 | ˜8 | 100 | 120 |
| IT-50 | 30% Co-polymer | 10 | 74.2 | 70-80 |
| | (acrylic acid 26.25% and butyl | | | |
| | acrylate 3.75%) | | | |
| IT-51 | 43.75% Co-polymer | 10 | 70.8 | 70-80 |
| | (acrylic acid 38.25% and butyl | | | |
| | acrylate 5.5%) | | | |
| IT-52 | 33.33% Co-polymer | 10 | 85.5 | 68-109 |
| | (acrylic acid 29.33% and butyl | | | |
| | acrylate 4%) | | | |
| IT-OS-38-17 | 30% Co-polymer | 12 | 95.5 | 67 |
| | (acrylic acid: butyl acrylate | | | |
| | 24:1) | | | |
| IT-56 | 33.3% polymer (acrylic acid) | 10 | 91.9 | 85 |

HPLC = High Performance Liquid Chromatography assay;
ND = not done

The results in Table 5 show that, when the anti-fungal agent itraconazole, which is an insoluble compound, is surrounded by an amphiphilic polymer, the resulting inclusion complex is hydrophilic. Using the matching technique described in the instant application, itraconazole is rendered hydrophilic when surrounded by various polymers which meet the matching parameters such as thermodestructed starch combined with H₂O₂ and PEG modification, alginate, and chitosan. The results show that 5% thermodestructed starch+1.25% H₂O₂+1.25% PEG combined with 5 mg/ml of iutraconazole resulted in an ALV of 382 nm; 5% thermo-destructed starch+0.625% H₂O₂+1.25% PEG combined with 5 mg/ml of itraconazole resulted in an ALV of 640 nm; 5% thermodestructed starch+1% H₂O₂+2% PEG combined with 5 mg/ml of itraconazole resulted in an ALV of 414 nm; 5% thermodestructed starch+1% H₂O₂+1% PEG combined with 5 mg/ml of itraconazole resulted in an ALV of 793 nm; 2% alginate combined with 20 mg/ml of itraconazole resulted in an ALV of 180 nm, and when combined with 10 mg/ml of itraconazole resulted in an ALV of 180 nm; 1% chitosan (Fluka) combined with ˜8 mg/ml iitraconazole resulted in an ALV of 120 nm. These results show that using the teachings of the specification (lipophilic/amphiphilic polymer matching technique), the insoluble anti-fungal agent itraconazole can be surrounded by various amphiphilic polymers (i.e. thermodestructed starch combined with H₂O₂ and PEG, alginate, and chitosan) to render the resulting inclusion complex hydrophilic in water.

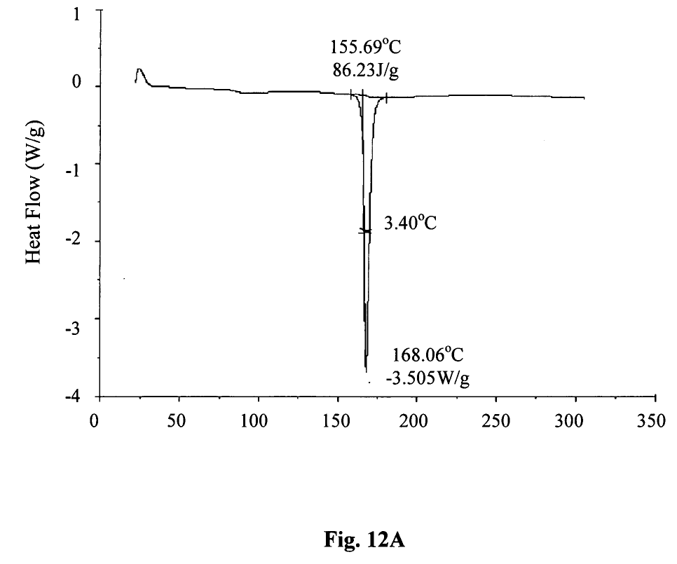

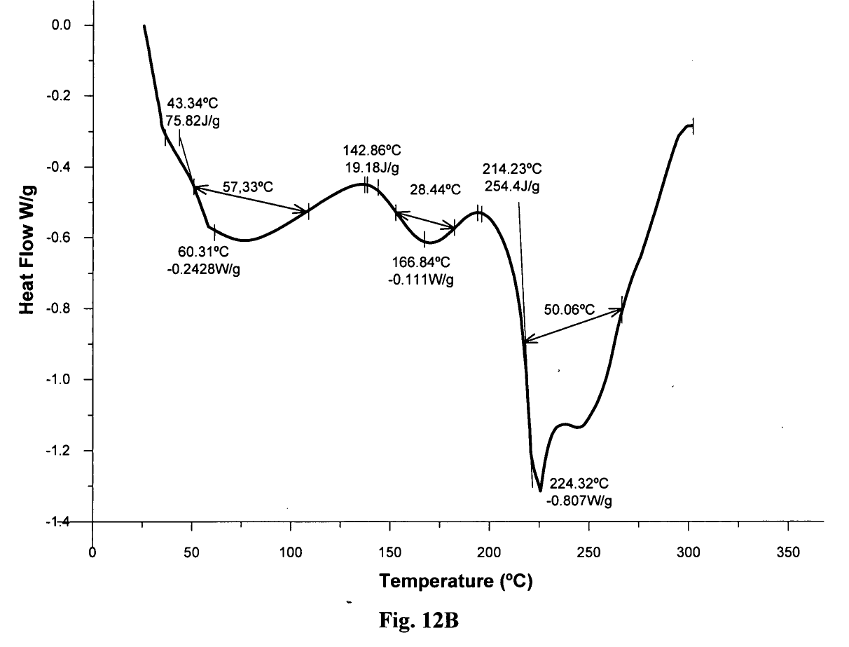

Differential scanning calorimetry (DSC) was done with a TA Instruments 2010 module and a 2100 System Controller to study the crystallinity of complexes. Prior to analysis, the samples are sealed in alodined aluminum DSC pans. The tests are done at a scan rate of 10 degrees/minute, from −50 to 200° C. FIGS. 12A-B provide illustrations of itraconazole crystals and the itraconazole complexes prepared in experiment IT-56 (see Table 5), respectively. While itraconazole crystals melt at the characteristic melting point, itraconazole complexes do not melt at the characteristic point.

### Example 12

### Physical Measurements and Characteristics of Various paclitaxel hydrophilic Inclusion Complexes

Inclusion complexes of the anticancer paclitaxel were prepared according to the method described in Example 1, in which paclitaxel was dissolved in methyl acetate or dichloromethane and the polymer was gelatin of different molecular weights with or without the addition of vitamin B12. Polyvinyl-pyrrolidone (PVP or povidone, e.g. Kollidon™ ) or polystyrene sulfonic acid can be added to increase solubilization of paclitaxel. Polystyrene sulfonic acid can also be used alone to solubilize paclitaxel.

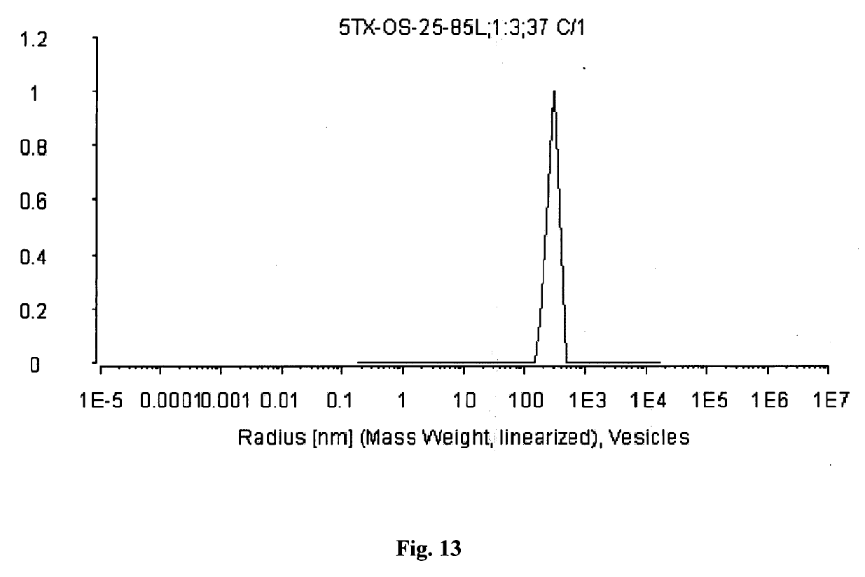

Table 6 below shows the properties of various such paclitaxel hydrophilic inclusion complexes. FIG. 13 illustrates the size distribution of nano-particles comprising paclitaxel hydrophilic inclusion complexes within gelatin (70-100 kD, 1 mg/ml vitamin B12) (# 25-85) having a size of approximately 179 nm.

TABLE 6
Properties of paclitaxel hydrophilic inclusion complexes in gelatin

| | | B₁₂ Conc. | Max | |
|---|---|---|---|---|
| | | in polymer | paclitaxel | Particle |
| | | solution | conc | Size |
| Exp. | Polymer (MW; mg/ml) | (mg/ml) | (mg/ml) | (nm) |
| 5TX- | Gelatin | 1 | 0.872 | 179 |
| OS-25- | (70-100 kD; 25 mg/ml) | | | |
| 85 | | | | |
| 5TX- | Gelatin | 0 | 0.646 | 117 |
| OS-25- | (70-100 kD; 25 mg/ml) | | | |
| 76 | | | | |
| 5TX- | Gelatin | 1 | 3.781 | 129 |
| OS-25- | (70-100 kD; 3 mg/ml) | | | |
| 89 | | | | |
| 5TX- | Gelatin | 0 | 0.925 | 186 |
| OS-25- | (70-100 kD; 25 mg/ml) | | | |
| 80 | | | | |
| 5TX- | Gelatin | 0.025 | 6.35 | 297 |
| OS-25- | (70-100 kD; 6 mg/ml) | | | |
| 119 | | | | |
| 5TX- | Gelatin | 0 | 0.8 | ND |
| OS-9- | (250 kD; 5 mg/ml) | | | |
| 147 | | | | |
| 5TX- | Gelatin | 0 | 0.05 | ND |
| OS-9- | (250 kD; 5 mg/ml)* | | | |
| 143 | | | | |
| 5TX- | Hydrolyzed gelatin | 0 | 0.066 | ND |
| OS-9- | (15 kD; 25 mg/ml) | | | |
| 116 | | | | |
| 5TX- | Gelatin | 0 | 0.92 | ND |
| OS-25- | (70-100 kD; 100 mg/ml)** | | | |
| 35 | | | | |

*and 20 mg/ml Kollidon (2000-3000 kD);
**and 111 mg/ml poly (4-styrenesulfonic acid);
ND = not done.

The results in Table 6 show that, when the anti-cancer agent paclitaxel, which is an insoluble compound, is surrounded by an amphiphilic polymer, the resulting inclusion complex is hydrophilic. Using the matching technique taught by the instant application, paclitaxel, at various concentrations (0.872 mg/ml, 0.646 mg/ml, 3.781 mg/ml and 0.925 mg/ml, for example) is rendered hydrophilic when surrounded by the polymer gelatin (optionally with added vitamin B12 excipient) which meets the matching parameters described in the instant application

### Example 13

### Physical Measurements and Characteristics of Various donepezil hydrophilic Inclusion Complexes

Inclusion complexes of donepezil hydrochloride were prepared according to the method described in Example 1, in which donepezil hydrochloride was dissolved in methyl acetate or dichloromethane and the polymers were modified corn starch, alginate, and sodium starch glycolate.

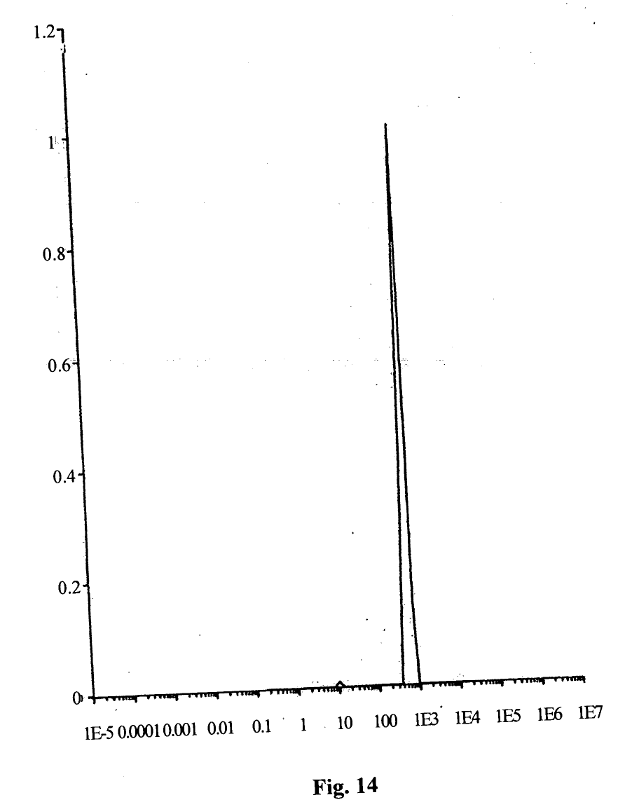

Table 7 below shows the properties of various such donepezil hydrochloride hydrophilic inclusion complexes. FIG. 14 illustrates the size distribution of nano-particles comprising donepezil hydrochloride hydrophilic inclusion complexes within modified corn starch (#LG-7-51) having a size of approximately 600 nm.

TABLE 7
Properties of donepezil hydrophilic inclusion complexes

| | Polymer | Drug | | HPLC After | ALV | | |
|---|---|---|---|---|---|---|---|
| Exp. | (name/%) | (%) | pH | dry % | nm | X-Ray | DSC |
| IC-130 | 2% Alginate | 2 | 5.2 | 97 | ND | Amorphous | Amorphous |
| | (Kelton) LV | | | | | | |
| LG-7- | 2% Na Starch | 1 | 5.5 | 80 | ND | ND | Amorphous |
| 38 | Glycolate | | | | | | |
| | (Explotab) | | | | | | |
| LG-7- | 1% Alginate | 1 | 5 | 103 | ND | ND | Amorphous |
| 44 | (Kelton) LV | | | | | | |
| LG-7- | 2% Corn Starch | 1 | 5 | 104 | 600 | Amorphous | ND |
| 51 | pregelatinized, | | | | | | |
| | modified | | | | | | |
| | (PureCote ™) | | | | | | |
| | B-793 | | | | | | |

HPLC = High Performance Liquid Chromatography assay;
ND = not done.

### Example 14

### Oral Absorption of Nano-Sized, Water-Soluble Particles of azithromycin and azithromycin Compositions

The oral absorption of water-soluble nano-sized particles comprising inclusion complexes of 1% azithromycin and 1% chitosan was studied in a preclinical model involving rats in comparison to a composition containing the commercially-available azithromycin (Azenil), in order to assess the contribution of the physical form for enabling absorption.

Azithromycin (50 mg/kg) is administered to male Sprague-Dawley rats (groups of 5), 250-280 g, by a feeding tube. At fixed times of administration (between 1-24 hours), blood samples are collected, and sera are prepared for analysis. At the end of the study, all rats are sacrificed by an IP overdose of pental (100 mg/kg).

Drug concentrations in rat serum (0.1 ml) are determined by LC-MS. The samples and calibration curve are prepared as follows: fifty (50) μl of sample are mixed with 50 μl of control serum to obtain a total volume of 100 μl of serum. The diluted samples are extracted with methyl tert-butyl ether, followed by evaporation and reconstitution in 40% aqueous acetonitrile. Analysis is performed by LC-MS, using atmospheric pressure electrospray ionization in the positive mode and an Agilent 1100 HPLC system. The azithromycin concentration is quantified by comparison with a calibration curve in the range from 20 to 2000 ng/ml, that is prepared using blank rat serum spiked with azithromycin. A plot of the concentrations (not shown) is used to determine the timing of the maximal concentration (Cₘₐₓ) and to assess the total absorption of the drug (as reflected by the area under the curve (AUC).

A summary of the main pharmacokinetic findings is presented in Table 8. These findings demonstrate that nano-sized, water-soluble particles having the same amount of azithromycin as Azenil, elevate the maximal concentration (Cₘₐₓ) obtained and the total amount of azithromycin absorbed (as reflected by the AUC). In addition, the concentration in the lung is particularly elevated, while the concentrations in other organs are increased to a less extent. Furthermore, there is no change in time at which the maximal concentration is reached.

TABLE 8
Comparison of pharmacokinetic parameters of azithromycin nano-
sized, water-soluble particles and Azenil

| | | | AUC | Lung, | Liver, | Kidney, | Heart, |
|---|---|---|---|---|---|---|---|
| | Tₘₐₓ | Cₘₐₓ | serum | 24 h | 24 h | 24 h | 24 h |
| SoluAzi | 2 | 1.56 | 7.4 | 13.7 | 28.5 | 36 | 1.49 |
| #55* | | | | | | | |
| Azenil | 2 | 0.91 | 4.2 | 6.9 | 18.7 | 31.6 | 1.26 |
| (control) | | | | | | | |
| Percent of | | 71 | 76 | 98 | 52 | 14 | 18 |
| increase | | | | | | | |

*a lot containing 1% azithromycin and 1% chitosan

The stability of azithromycin particles, following compression, and their compatibility with tablet excipients are assessed by comparing azithromycin absorption with that of the complexes prior to tablet preparation. Tablets are prepared following lyophilization of complexes and subsequent mixture with standard acceptable excipients. The tablets are formed by application of pressure up to 1 ton/cm². Prior to administration to rats, the tablets are dissolved in water. Then, azithromycin (50 mg/kg) is administered to male Sprague-Dawley rats (groups of 5), 250-280 g, by a feeding tube. Pharmacokinetic studies involving oral administration are done as described above.

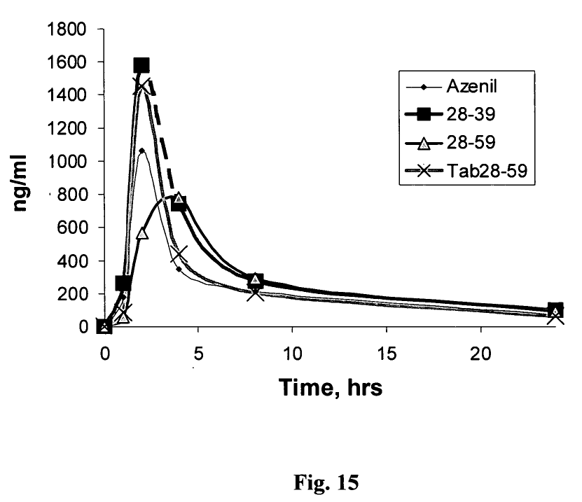

Drug concentrations in rat serum are analyzed as described above. Plots of the serum concentrations are presented in FIG. 15. In this figure, Azenil is a marketed commercial formulation of azithromycin, while lots 28-39 and 28-59 are solutions of nano-sized, water-soluble particles comprising 1% azithromycin complexes with 1% chitosan, and Tab 28-59 is a tablet prepared from lot 28-59, dissolved in water immediately prior to administration. It is clear from this figure that the maximal concentration of azithromycin is generally reached at the same time for all of the preparations. However, absorption of azithromycin from the particles is always greater than that of the commercial formulation. Thus, as demonstrated above, enhanced absorption is apparently associated with formulations comprising the water-soluble nano-sized particles. Furthermore, the calculated area under the curve for the tablet is only about 10% less that that of the solutions comprising the water-soluble nano-sized particles. Therefore, the steps taken to prepare tablets do not adversely affect the nano-sized particles.

### Example 15

### Oral Absorption of Nano-Sized, Water-Soluble Particles of itraconazole

The oral absorption of itraconazole nano-sized, water-soluble particles comprising itraconazole inclusion complexes with copolymer of acrylic acid and butyl acrylate (#IT-50, Table 5) was studied in a preclinical model involving rats and compared with oral absorption of itraconazole in compositions comprising itraconazole mixed by vortex with polyacrylic acid, which do not form nano-particles, in order to assess the contribution of the physical form for enabling absorption.

Itraconazole (50 mg/kg) is administered to male Sprague-Dawley rats (groups of 5), 250-280 g, by a feeding tube. At fixed times of administration (between 1-24 hours), blood samples are collected, and sera are prepared for analysis. At the end of the study, all rats are sacrificed by an IP overdose of pental (100 mg/kg).

Drug concentrations in rat serum (0.1 ml) are determined by HPLC using a method essentially as described by Yoo et al. (2002) Arch Pharm Res 25:387-391. The samples and calibration curve are prepared as follows: samples are mixed with an equal volume of acetonitrile to obtain a total volume of 400 μl. KCl is added to the samples for protein precipitation, and itraconazole, in the subsequent supernatant, is applied to a Merck HPLC system. The itraconazole concentration is quantified by comparison with a calibration curve in the range from 20 to 1000 ng/mL, that is prepared using blank rat serum spiked with itraconazole. A plot of the concentrations (not shown) is used to determine the timing of the maximal concentration (Cₘₐₓ) and to assess the total absorption of the drug (as reflected by the area under the curve (AUC).

A summary of the main pharmacokinetic findings is presented in Table 9. These findings demonstrate that, administration of nano-sized, water-soluble particles having the same amount of intraconazole, doubles the elevated maximal blood concentrations (Cₘₐₓ) of both itraconazole and its active hydroxylated metabolite (hydroxyitraconazole) and the total amount of itraconazole absorbed is increased, as reflected by the areas under the curve (AUC) of both itraconazole and its active hydroxylated metabolite.

TABLE 9
Comparison of pharmacokinetic parameters of itraconazole as water-
soluble particles (IT-50) and as mechanical mixture (MIX) with polymer

| | Itraconazole | | OH-itraconazole | |
|---|---|---|---|---|
| | IT-50 | MIX | IT-50 | MIX |
| Cₘₐₓ | 0.46 | 0.22 | 0.72 | 0.38 |
| Tₘₐₓ | 4 | 4 | 4 | 4 |
| AUC | 6.9 | 5.8 | 13.3 | 9.5 |

1. A hydrophilic dispersion of nano-sized particles comprising:
(a) a water-insoluble or water-soluble active compound, wherein said active compound is selected from the group consisting of a macrolide antibiotic, donepezil hydrochloride, an azole compound and a taxane; and
(b) an amphiphilic polymer which wraps said active compound in a non-crystalline manner to form a nano-sized molecular entity in which no valent bonds are formed.

2. The hydrophilic dispersion of claim 1, wherein said active compound is wrapped within said amphiphilic polymer via non-valent interactions between said polymer and said active compound such that said interactions fixate said active compound within said polymer.

3. The hydrophilic dispersion of claim 2, wherein said non-valent interactions include electrostatic forces, Van der Waals forces, coordinative bonds and hydrogen bonds.

4. The hydrophilic dispersion of claim 1, wherein said active compound wrapped in said amphiphilic polymer is fixated within said polymer.

5. The hydrophilic dispersion of claim 1, wherein said nano-sized molecular entity is substantially spherical.

6. The hydrophilic dispersion of claim 1, wherein said amphiphilic polymer is selected from the group consisting of polysaccharides, polyacrylic acid and its derivatives and copolymers thereof, polymethacrylic acid and its derivatives and copolymers thereof, polyethylene imine and its derivatives, polyethylene oxide and its derivatives, polyvinyl alcohol and its derivatives, polyisoprene derivatives, polybutadiene derivatives and gelatin.

7. The hydrophilic dispersion of claim 6, wherein said amphiphilic polymer is a polysaccharide selected from the group consisting of starch, chitosan and an alginate.

8. The hydrophilic dispersion of claim 7, wherein said starch is modified to increase its hydrophilicity, or to reduce its branching, or both.

9. A nano-dispersion of claim 1, of water-soluble and stable nano-sized particles comprising hydrophilic inclusion complexes consisting essentially of an active compound surrounded by and entrapped within an amphiphilic polymer, wherein said active compound is in a non-crystalline state and said inclusion complex is stabilized by non-valent interactions between the active compound and the surrounding amphiphilic polymer, and wherein said inclusion complex is selected from the group consisting of:
(i) an inclusion complex wherein the active compound is a macrolide antibiotic and the amphiphilic polymer is a polysaccharide or polyvinyl alcohol;
(i) an inclusion complex wherein the active compound is donepezil hydrochloride and the amphiphilic polymer is a polysaccharide;
(iii) an inclusion complex wherein the active compound is an azole compound and the amphiphilic polymer is selected from the group consisting of a polysaccharide, polyacrylic acid, a copolymer of polyacrylic acid, polymethacrylic acid and a copolymer of polymethacrylic acid; and
(iv) an inclusion complex wherein the active compound is a taxane and the amphiphilic polymer is gelatin.

10. The nano-dispersion of claim 9, wherein the nano-particles comprise inclusion complexes in which the active compound is a macrolide antibiotic and the amphiphilic polymer is a polysaccharide.

11. The nano-dispersion of claim 10, wherein the macrolide antibiotic is erythromycin, clarithromycin, or azithromycin.

12. The nano-dispersion of claim 10, wherein said polysaccharide is starch or starch modified to increase its hydrophilicity, or to reduce its branching, or both.

13. The nano-dispersion of claim 12, wherein said starch is modified by one or more of the following treatments: acid hydrolysis, reaction with polyethylene glycol or hydrogen peroxide, or thermal treatment.

14. The nano-dispersion of claim 10, wherein the nano-particles comprise inclusion complexes in which the active compound is erythromycin and the amphiphilic polysaccharide is starch modified by one or more of the following treatments: acid hydrolysis, reaction with polyethylene glycol or hydrogen peroxide, or thermal treatment.

15. The nano-dispersion of claim 10, wherein the nano-particles comprise inclusion complexes in which the active compound is clarithromycin and the amphiphilic polysaccharide is selected from the group consisting of chitosan, alginate, and starch that has been modified by one or more of the following treatments: acid hydrolysis, reaction with polyethylene glycol or hydrogen peroxide, or thermal treatment.

16. The nano-dispersion of claim 10, wherein the nano-particles comprise inclusion complexes in which the active compound is azithromycin and the amphiphilic polysaccharide is chitosan or propylene glycol alginate.

17. The nano-dispersion of claim 10, wherein the nano-particles comprise inclusion complexes in which the active compound is azithromycin and the amphiphilic polymer is polyvinyl alcohol (PVA).

18. The nano-dispersion of claim 9, wherein the nano-particles comprise inclusion complexes in which the active compound is donepezil hydrochloride and the amphiphilic polymer is a polysaccharide.

19. The nano-dispersion of claim 18, wherein said polysaccharide is selected from the group consisting of alginate, sodium starch glycolate and pregelatinized modified starch.

20. The nano-dispersion of claim 9, wherein the nano-particles comprise inclusion complexes in which the active compound is an azole compound and the amphiphilic polymer is selected from the group consisting of a polysaccharide, polyacrylic acid, a copolymer of polyacrylic acid, polymethacrylic acid and a copolymer of polymethacrylic acid.

21. The nano-dispersion of claim 20, wherein the azole compound is an imidazole or triazole compound for human or veterinary application or for use in the agriculture.

22. The nano-dispersion of claim 21, wherein the azole compound is an azole fungicide selected from the group consisting of terconazole, itraconazole, fluconazole, clotrimazole, miconazole, econazole, ketoconazole, tioconazole, isoconazole, oxiconazole, and fenticonazole.

23. The nano-dispersion of claim 21, wherein the azole compound is a nonsteroidal antiestrogen selected from the group consisting of letrozole, anastrozole, vorozole, and fadrozole.

24. The nano-dispersion of claim 21, wherein the azole compound is an azole fuingicide useful in the agriculture selected from the group consisting of bitertanol, cyproconazole, difenoconazole, epoxiconazole, fluquinconazole, flusilazole, flutriafol, hexaconazole, metconazole, myclobutanil, penconazole, propiconazole, tebuconazole, triadimefon, triadimenol, and triticonazole, imazalil, prochloraz, and triflumizole.

25. The nano-dispersion of claim 21, wherein the azole compound is a nonfungicidal azole for use in the agriculture selected from the group consisting of azocyclotin, paclobutrazole, carfentrazone, isazophos, and metazachlor.

26. The nano-dispersion of claim 20, wherein the amphiphilic polysaccharide is selected from the group consisting of chitosan and starch that has been modified by one or more of the following treatments: acid hydrolysis, reaction with polyethylene glycol or hydrogen peroxide, or thermal treatment.

27. The nano-dispersion of claim 20, wherein the amphiphilic polymer is polyacrylic acid or a copolymer of acrylic acid with butyl acrylate.

28. The nano-dispersion of claim 20, wherein the azole compound is itraconazole and the amphiphilic polymer is selected from the group consisting of polyacrylic acid, a copolymer of acrylic acid with butyl acrylate, chitosan, and starch that has been modified by one or more of the following treatments: acid hydrolysis, reaction with polyethylene glycol or hydrogen peroxide, or thermal treatment.

29. The nano-dispersion of claim 9, wherein the nano-particles comprise inclusion complexes in which the active compound is a taxane and the amphiphilic polymer is gelatin.

30. The nano-dispersion of claim 29, wherein the taxane is paclitaxel, docetaxel or a semy-synthetic derivative of a taxane.

31. The nano-dispersion of claim 29, wherein an agent selected from the group consisting of vitamin B12, polyvinylpyrrolidone and poly(4-styrenesulfonic acid) is added to the gelatin.

32. A process for preparation of a nano-dispersion of claim 1, the process comprising the steps of:
(i) preparing a molecular solution of the amphiphilic polymer in water;
(ii) preparing a molecular solution of the active compound in an organic solvent, wherein said active compound is selected from the group consisting of a macrolide antibiotic, donepezil hydrochloride, an azole compound and a taxane;
(iii) dripping the cold solution of the active compound (ii) into the heated polymer solution (i) at a temperature 5 to 10° C. above the boiling point of the organic solvent, under constant mixing; and
(iv) removing the organic solvent thus obtaining the nano-dispersion comprising the nano-particles consisting of the inclusion complexes wherein said active compound is wrapped within said amphiphilic polymer via non-valent interactions.

33. A stable pharmaceutical composition comprising a nano-dispersion of claim 9 and a pharmaceutically acceptable carrier.

34. The stable pharmaceutical composition of claim 33 for oral administration.

35. The stable pharmaceutical composition of claim 34 in liquid or solid form.

36. The stable pharmaceutical composition of claim 35 in the form of tablets.

37. The stable pharmaceutical composition of claim 33 for treatment of bacterial infections comprising a nano-dispersion of water-soluble nano-particles comprising an inclusion complex wherein the active compound is a macrolide antibiotic selected from the group consisting of erythromycin, clarithromycin and azithromycin and the amphiphilic polymer is a polysaccharide or polyvinyl alcohol.

38. The stable pharmaceutical composition of claim 33 for treatment of dementia and Alzheimer's disease comprising a nano-dispersion of water-soluble nano-particles comprising an inclusion complex wherein the active compound is donepezil hydrochloride and the amphiphilic polymer is a polysaccharide.

39. The stable pharmaceutical composition of claim 33 for treatment of fungal infections comprising a nano-dispersion of water-soluble nano-particles comprising an inclusion complex wherein the active compound is an azole fungicide and the amphiphilic polymer is selected from the group consisting of a polysaccharide, polyacrylic acid, a copolymer of polyacrylic acid, polymethacrylic acid and a copolymer of polymethacrylic acid.

40. The stable pharmaceutical composition of claim 39 wherein the azole fungicide is itraconazole and the amphiphilic polymer is selected from the group consisting of polyacrylic acid, a copolymer of acrylic acid with butyl acrylate, chitosan, and starch that has been modified by one or more of the following treatments: acid hydrolysis, reaction with polyethylene glycol or hydrogen peroxide, or thermal treatment.

41. The stable pharmaceutical composition of claim 33 for treatment of estrogen-responsive breast tumors comprising a nano-dispersion of water-soluble nano-particles comprising an inclusion complex wherein the active compound is a nonsteroidal antiestrogen azole selected from the group consisting of letrozole, anastrozole, vorozole and fadrozole.

42. The stable pharmaceutical composition of claim 33 for treatment of cancer comprising a nano-dispersion of water-soluble nano-particles comprising an inclusion complex wherein the active compound is a taxane and the amphiphilic polymer is gelatin.

43. The stable pharmaceutical composition of claim 42 for treatment of cancer wherein the taxane is paclitaxel.
